(19) United States
(12) Patent Application Publication
(10) Pub. No.: US 2014/0206072 A1
(43) Pub. Date: Jul. 24, 2014

(54) METHODS AND SYSTEM FOR OBTAINING ALGAL BIOMASS AND CULTIVATION MEDIUM

(71) Applicants: Ana Marta Severino Do Rosario De Quintanilha Dos Santos, 8934 CJ Leeuwarden (NL); Packo Petrus Lamers, 8934 CJ Leeuwarden (NL); Marcel Janssen, 8934 CJ Leeuwarden (NL)

(72) Inventors: Ana Marta Severino Do Rosario De Quintanilha Dos Santos, 8934 CJ Leeuwarden (NL); Packo Petrus Lamers, 8934 CJ Leeuwarden (NL); Marcel Janssen, 8934 CJ Leeuwarden (NL)

(21) Appl. No.: 14/233,918

(22) PCT Filed: Jul. 18, 2012
(86) PCT No.: PCT/NL2012/000054

§ 371 Date: Mar. 13, 2014

(30) Foreign Application Priority Data
Jul. 26, 2011 (NL) 2007181

(51) Int. Cl. C12N 1/12; C12M 1/34

(52) U.S. Cl. CPC C12N 1/12; C12M 41/26
USPC 435/257.1; 435/303.1

(57) ABSTRACT

The present invention relates to a method for obtaining algal biomass, a system for obtaining algal biomass and a cultivation medium for cultivating algae. The method comprises the step of culturing algae in a culturing medium having a pH above 8 such that lipid accumulation and/or carbohydrate accumulation is increased, wherein the medium is a saline medium. Preferably, the pH of the medium is in the range of 8-11, preferably 8.5-11, more preferably 9-11 and most preferably 10-11.

The present invention relates to a method for obtaining algal biomass. In particular, the invention relates to obtaining algal biomass, e.g. lipids, such as fatty acids comprised by algal cells, by cultivating micro-algae. For example, biomass from (micro-)algae is obtained for use as biofuels, fertilizer or nutrition.

It is known in the art to cultivate (micro-)algae for obtaining algal biomass for instance for use as biofuels, fertilizer, feedstock or nutrition. For example, (micro-) algae are cultivated to obtain lipids, proteins, polysaccharides and certain vitamins. The yield of such substances is limited in conventional methods. Specifically, lipid-rich microalgae are one of the most promising organisms for a sustainable production of biofuels. Yet, there are still some challenges with respect to process optimization. One of those challenges is to improve the lipid yields on light by maximization of biomass and lipid productivities. Maximal productivity of photoautotrophic microalgae is attainable when light is the only limitation for biomass growth, which requires that all necessary nutrients are present and that temperature, salinity and pH are optimal. Under such optimal conditions, the efficiency with which light energy is used for microalgae growth depends largely on the incident light intensity, the optical path length and the biomass density. Microalgal biofuels production may require natural sunlight and photobioreactors with a non-adjustable optical path length. Hence, the most suitable parameter that can be controlled to achieve an optimal photosynthetic efficiency throughout the entire production period is the biomass density. Such control is readily attainable by adjusting the dilution rate of the system. Implementing such a control strategy requires detailed knowledge on the relations between incident light intensity, biomass density and photosynthetic efficiency. Another matter concerning the existing microalgal biofuels production technologies is the high energy input for the transfer of carbon dioxide (CO₂) from gas to liquid. A promising option for reducing this energy cost in photobioreactors is to optimize the CO2 transfer in a separate gas transfer unit at elevated pH. In this way, the growth medium could be saturated with dissolved bicarbonate (HCO₃⁻) before entering the cultivation system. Besides for a 2-stage process, working at high pH is also advantageous for traditional single-stage microalgae cultivation processes. CO₂ dissolution can be enhanced at high pH by direct reaction of CO₂ with hydroxide (OH—) in the liquid boundary layer surrounding the gas bubbles.

This results in a fast formation of HCO₃⁻ which serves as an extra carbon carrier over the boundary layer. Bicarbonate would then serve as the major source of inorganic carbon and less energy would need to be invested in CO2 gas-liquid transfer inside the photobioreactor.

The goals of the present invention, amongst other goals, are to overcome or reduce the above mentioned disadvantages and to provide an improved method for obtaining algal biomass by cultivating algae.

This objective is achieved with the method according to the invention, the method comprising the step of culturing algae in a culturing medium having a pH above 8 such that lipid accumulation and/or carbohydrate accumulation in the algal biomass is increased, wherein the medium is a saline medium. In the context of the present invention the culturing medium is to be understood as a culturing aqueous medium suitable for pH measurement.

Surprisingly, cultivating algae, preferably micro-algae, in a culturing medium having a pH above 8 alters the composition of the algal biomass. The lipid content and/or the carbohydrate content of the algae increases. In other words the ratio of lipids and/or the ratio of carbohydrates in the algal biomass increase(s). Furthermore, the rate of this increase is relatively high. In other words, the ratio of accumulated lipids and/or carbohydrates in the algal biomass is increased, and this higher ratio is achieved in a relative short period of time.

For example, the algae are cultivated in a medium with a substantially constant pH level above 8.0. Alternatively, the algae are grown in a first step at conventional pH levels until a constant growth rate has been established. In a next step the pH of the medium is elevated to achieve lipid and/or carbohydrate accumulation.

A further advantage is that by cultivating algae at a pH higher than 8.0, the CO₂ transfer to the medium is improved. When cultivating algae according to conventional methods, substantial energy is required for the supply of carbon dioxide (CO₂) to large-scale cultivation systems by means of gas transfer, which is a big restriction for the potential uses of these systems. During (micro-) algal cultivation, CO₂ is usually introduced to the system by CO₂ enrichment of the inlet air flow, but it is also envisioned to use CO₂-rich gasses as for example flue gas. However, a considerable amount of CO₂ which fails to be transferred into the liquid phase will still be wasted to the effluent gas.

By using a medium with a pH above 8, the CO₂ uptake of a medium is increased.

In a fluid with dissolved CO₂, an equilibrium exists between dissolved bicarbonate (HCO³⁻) and CO₂ and, in the case of a high pH, also dissolved carbonate (CO₃²⁻). When (micro-)algae take up CO₂ from the fluid, CO₂ is replenished by dissociation of bicarbonate in OH⁻ and CO₂. This increases the pH of the fluid. If no CO₂ is added to compensate for the carbon dioxide uptake by algae, the pH will continue to increase, even above 11 and finally only carbonate will remain in the fluid.

Working at conventional pH levels (<8), the supplied carbon dioxide gas has to react with water (H₂O) to form H₂CO₃, which subsequently (partially) dissociates to H⁺ and HCO³⁻. This is a relatively slow chemical reaction.

In the method according to the invention, due to the increased pH level, the CO₂ can react directly with the OH⁻ to form HCO³⁻.

This reaction is faster than the above described reaction involving dissociation of H₂CO₃⁻. When using a pH above 8 according to the method of the invention, diffusion of HCO³⁻ at the fluid/gas boundary layer of the gas bubbles contributes to the transfer of the substances. In contrast, in conventional methods with a lower pH, only CO₂ diffuses at the boundary layer.

Due to the relative fast transfer of CO₂ to the medium in the method according to the invention, the CO₂ gas can be supplied to the medium at a lower rate. This significantly reduces energy consumption.

Furthermore, the cultivation medium can comprise a relatively large concentration of inorganic carbon as bicarbonate (HCO³⁻), which provides a CO₂ source. Therefore, less CO₂ has to supplied to the medium by an external source, such as a gas supply.

For example, in the method according to the invention, bicarbonate is added to the medium prior to supplying the medium to a cultivation system. The energy necessary for CO₂ transfer from the gas to the liquid phase could thus be reduced by optimizing CO₂ transfer in a separate gas transfer unit at elevated pH (>8.0).

According to the invention, cultivation can be batch-wise or continuous.

For example, lipids comprise fatty acids. For example, carbohydrates comprise polysaccharides such as starch.

In a preferred embodiment according to the invention, the pH of the medium is in the range of 8-11, preferably 8.5-11, more preferably 9-11 and most preferably 10-11. In the context of the present invention, the pH values and ranges given are to be understood as an indication of the activity of the (solvated) hydrogen ion. The abbreviation p[H], which measures the hydrogen ion concentration is called pH. Solutions with a pH less than 7 are said to be acidic and solutions with a pH greater than 7 are basic or alkaline. The pH scale is traceable to a set of standard solutions whose pH is established by international agreement. Primary pH standard values are determined using a concentration cell with transference, by measuring the potential difference between a hydrogen electrode and a standard electrode such as the silver chloride electrode.

Measurement of pH for aqueous solutions can be done with a glass electrode and a pH meter, or using indicators. In the method of the present invention, the pH is above 8, advantageously above 8.5, more advantageously above 9, most advantageously above 9.5. In the context of the present invention, the pH is below 12, advantageously below 11.5, more advantageously below 11. The pH can have any values between 8.1 and 11.

Preferably the pH of the medium lies between 8.1 or 8.2 or 8.3 or 8.4 or 8.5 and 11 or 10.9 or 10.8 or 10.7 or 10.6 or 10.5.

Experiments conducted by the inventors show good results using in particular a pH of about 10. The term “about” is to be understood as within the experimental measurement error margin. Accordingly, all the values and ranges in the context of the present invention may vary around one value or range within a few tenth of pH unit, depending on the precision of the measuring instrument.

In a preferred embodiment the algal biomass comprises algal cells, preferably lipids comprised by the algal cells.

For example, lipids are obtained for use as biofuels or feedstock. Surprisingly, in particular lipid accumulation is increased in algal biomass when cultivating algae in a medium having a pH above 8, preferably equal to, or above 8.2, such as equal to, or above 8.5, equal to, or above 9, equal to, or above 9.5, equal to, or above 10. The pH can have any values chosen from the group 8.2, 8.5, 9, 9.5, 10, 10.5 and 11. The final concentration of lipids is higher than in conventional methods and this level is achieved quicker.

According to a further preferred embodiment, the algae are lipid-rich algae. Certain types of algae are known for their intrinsic high level of lipids. By cultivating these algae using the method of the invention, the lipid content of the algae can be increased even further.

In an embodiment according to the invention, the algae comprise micro-algae, preferably freshwater micro-algae.

Preferably, the micro-algae are lipid-rich micro-algae, when the method is aimed at harvesting lipids from algae.

In fact, due to their high photosynthetic efficiency, high biomass productivity and fast growth rates in comparison to higher plants, lipid-rich micro-algae can be considered as potentially one of the most efficient biological oil producers.

In a preferred embodiment, the algae comprise algae of the species Neochloris oleoabundans.

Neochloris oleoabundans is capable of producing oil bodies composed of triacylglycerides (TAG) at a level of up to 80% of its total lipid composition. Most of those lipids are composed of saturated fatty acids, perfectly suitable for the production of biofuels.

Surprisingly, algae of the Neochloris oleoabundans species are able to accumulate lipids under high pH (>8.0) conditions. Furthermore, the lipid content of the algae increases as compared to cultivation in a lower pH medium.

In a preferred embodiment according to the invention, the medium is a saline medium. By “saline medium” is to be understood saline aqueous medium.

In the context of this invention, the term saline refers to the salt contents, excluding those salts influencing the hardness of the aqueous medium. In the context of the present invention, the hardness of the aqueous medium is determined by the concentration of multivalent cations in the water. Multivalent cations are cations (positively charged metal complexes) with a charge greater than 1+. Specifically, in the context of the present invention, the cations have the charge of 2+. Common cations found in hard water include Ca²⁺ and Mg²⁺. Accordingly, the saline aqueous medium according to the present invention is not to be understood as a medium comprising metal ions of the second column of the periodic table, such as in calcium salts and magnesium salts. Further, the saline aqueous medium in the context of the present invention excludes the salts which influence the pH of the medium, such as buffer aqueous medium, or such as aqueous solutions comprising anions such as bicarbonate or carbonate. According to this definition, the salinity will therefore be mainly determined by the concentration of alkali metal chloride salts, e.g. any chloride salt of metal chosen from the group consisting of lithium, sodium, potassium, rubidium, cesium and francium, in particular sodium chloride (NaCl) or potassium chloride (KCl).

Good results have been obtained by the method of the present invention when the salinity is in the range 0.7 to 7%, preferably 1% to 6%, more preferably in the range 2 to 5%, most preferably in the range 3% to 5%. By the term “salinity” is to be understood the weight % of salt relative to the total weight of aqueous medium.

Surprisingly, the growth rate of algae under high pH conditions turns out to be higher when using a saline aqueous medium than using, for example, a freshwater based medium.

In particular, an alkaline (pH>8.0) and saline aqueous medium is used to grow algae of the species Neochloris oleoabundans.

Preferably, the medium comprises seawater and/or artificial seawater.

This is in particular an advantage when cultivating these algae in a location where freshwater is scarce. Using the method of the invention seawater can be used instead of freshwater.

In a preferred embodiment of the method according to the invention, the cultivating medium comprises alkali metal chlorine salts, preferably NaCl, in a concentration providing the medium with a higher salinity than freshwater, preferably in a concentration of at least 120, 200, 270 or 400 mM.

Freshwater can be defined as water with less than 500 parts per million of dissolved salts.

Preferably, the sum of the concentrations of Na, CO₃²⁻ and HCO³⁻ is about 350-500 mM, more preferably 420 mM.

In a preferred embodiment, the pH of the culturing medium is regulated by equilibrium of carbon dioxide, bicarbonate and carbonate.

By culturing the algae in a medium comprising bicarbonate (HCO³⁻) and/or carbonate (CO₃²⁻), the alkalinity of the medium can be regulated by the distribution of the bicarbonate and carbonate species (HCO₃ and CO₃²⁻).

Bicarbonate and carbonate salts determine the buffering capacity of the medium. By providing them at high enough levels to obtain this buffer function, the necessary CO₂ for algal growth is provided, while preventing pH variations. Preferably, CO₂ is supplied to compensate for CO₂ uptake.

In a further embodiment the bicarbonate and carbonate ions are present in an amount sufficient to buffer the medium at a pH of at least 8, 8.2, 8.5, 9, 9.5, 10, 10.5 or 11.

For example, bicarbonate and carbonate is provided by adding NaHCO₃ and Na₂CO₃. For example bicarbonate and carbonate are each present in a concentration of at least 10, 30, 50, 60, 75, 90, 100, 150, 210, 240 or at least 270 mM. Advantageously, bicarbonate and carbonate anions are each present at a concentration in the range 10 to 150 mM, more advantageously, 50 to 100 mM. More advantageously, the concentration of bicarbonate or carbonate is 75 mM.

Preferably the medium is saturated with the bicarbonate and/or carbonate.

The CO₂ transfer can take place prior to supplying it to a system for growing algae, or during the growth stage. In traditional systems where CO₂ is supplied in the actual growth stage, the efficiency of CO₂ transfer can be increased by the method according to the invention, for example by operating the system at a pH of 8.5 or more, preferably a pH of 10 or more.

In a preferred embodiment, the carbon dioxide is supplied in a gas which comprises 0.01%-20% (v/v) carbon dioxide, preferably 0.038-20% (v/v), more preferably 0.038-4% (v/v).

For example, air is supplied which has a carbon dioxide content of about 0.038% (v/v). In another example, a gas comprising 0.01-4% carbon dioxide is supplied. In another example, a flue gas comprising 10-20% (v/v) carbon dioxide is supplied.

By recirculating the gas, the carbon dioxide content may decrease with respect to the initial carbon dioxide content.

In a preferred embodiment of the method according to the invention, the algae are cultivated under conditions of nutrient limitation.

It is known in the art that nutrient limitation can increase the lipid and/or carbohydrate content of micro-algae. For biofuel and nutritional applications, the lipid content of algae is of particular importance.

Surprisingly, the combination of a high pH and nutrient limitation results in an even more pronounced lipid accumulation and/or carbohydrate accumulation as compared to nutrient limitation alone.

In a preferred embodiment of the method according to the invention, the nutrient starvation conditions comprise nitrogen limitation, preferably nitrogen starvation.

For example, the method comprises providing a nitrogen-poor or nitrogen-free medium after a constant growth rate has been established.

Nitrogen limitation can be defined as the condition wherein the amount of nitrogen is a growth limiting factor. Nitrogen starvation can be defined as the condition wherein the amount of nitrogen is the dominant growth limiting factor.

In particular, the method according to the invention comprises the combination of cultivating algae of the Neochloris oleoabundans species in a cultivating medium having a pH above 8 and applying a nutrient limiting condition, preferably nitrogen limitation.

Experiments of the inventors show that at high pH and under nitrogen limiting conditions the most abundant fatty acids in Neochloris oleoabundans are C16:0, C18:1 and C18:2, which together account for more than 90% of the total fatty acid content. These fatty acids form a suitable feedstock for the production of biofuels, coatings and surface active agents. The fraction of these fatty acids that is present in triglycerides can also serve as a replacement of the traditional crop oil that is used in food industry.

Preferably the algae are of the species Neochloris sp., Neochloris oleoabundans, Ettlia sp. or Ettlia texensis.

Alternatively, the algae are of the species Scenedesmus obliquus, Chlorella vulgaris, Chlorella zofingiensis, Dunaliella tertiolecta, Dunaliella salina, Tetraselmis suecica, Parietochloris incise, Nannochloris sp., Nannochloropsis sp., Nannochloropsis oculata, Nannochloropsis salina, Isochrysis galbana, Porphyridium cruentum, Phaeodactylum tricornutum, Chaeotoceros muelleri, Chaeotoceros calcitrans, Chaeotoceros gracilis or Monodus subterraneus.

In a preferred embodiment of the method according to the invention, the cultivating medium comprises ions of a soluble salt, the concentration being such that no significant precipitation of the ions at the given pH level occurs, i.e. the saturation point is not reached.

Algal growth depends on an adequate supply of nutrients. These nutrients are usually highly soluble salts which dissociate in ions and are either present in high concentrations, as in the case of magnesium (Mg²⁺) and calcium (Ca²⁺) in seawater, or can be added in amounts sufficient to support good algal growth. At high pH (pH>8.0), especially divalent ions have the tendency to precipitate, with phosphate or hydroxide as insoluble salts. For example, the binding capacity of calcium to phosphate is pH dependent, increasing with elevated pH values. Also magnesium precipitates at higher pH, although at a lesser degree than calcium. Compounds which precipitate are no longer available for algal growth. For this reason, the concentrations of ions such as Ca²⁺, Mg²⁺ and phosphate (PO₄³⁻) should be kept as low as possible without limiting (micro-) algae growth.

In a preferred embodiment of the method according to the invention, the cultivating medium comprises Ca and Mg ions, wherein the concentration of Ca ions is between 0.001 and 0.15 mM and the ratio Ca:Mg is between 1:5 and 1:30, preferably 1:10 to 1:20, more preferably 1:15.

Experiments confirm that the amount of magnesium needed by algae is higher than the amount of calcium. Good results are obtained with a ratio of 1:15.

Preferably, Mg ions are provided in a concentration of 0.15 mM and Ca ions in a concentration of 0.01 mM. For example, the medium comprises 0.15 mM MgCl₂.6H₂O and 0.01 mM CaCl₂.2H₂O. Preferably the medium further comprises 4.4 mM KNO₃ and 0.32 mM polyphosphate. Preferably the medium is based on an marine medium, for example an f/2 medium.

In a preferred embodiment, the method according to the invention further comprises measuring the pH and/or the ion concentration.

Preferably the concentration of magnesium, calcium and/or phosphate is measured. Preferably, the method further comprises adding magnesium, calcium and/or phosphate when the respective measured concentration is below a certain threshold. This has the advantage that the concentration of the ions is kept constant. Additionally or alternatively, the pH level is monitored and the pH is adjusted when it is above or below a given threshold. Preferably the measurement of the ion concentration and/or pH level is performed using one or more sensors which are connected to a control unit, for performing the monitoring and regulation of ion concentrations and/or pH level automatically.

The invention further relates to a system for obtaining algal biomass using the method described above, comprising:

a container for holding the cultivating medium; and

means for regulating the pH level of the medium in the container to a level above 8, preferably above 8.5, 9, 9.5, 10, 10.5 or 11.

For example, the regulation means comprise a sensor for measuring the pH level.

Finally, the invention relates to a cultivation medium for cultivating algae using the method described above, having a pH above 8.0. Preferably the medium comprises carbonate and/or bicarbonate.

The same advantages and effects as described above in relation to the method for cultivating algae apply to the system and medium according to the invention.

Preferably the cultivation medium comprises Ca and Mg ions, the concentration of Ca ions being between 0.001 and 0.15 mM and the ratio Ca:Mg being between 1:5 and 1:30, preferably 1:10 and 1:20, more preferably 1:15.

Preferably the pH of the medium lies between 8 and 11, more preferably between 8.1 or 8.2 or 8.3 or 8.4 or 8.5 and 11 or 10.9 or 10.8 or 10.7 or 10.6 or 10.5.

Preferably, bicarbonate and carbonate is provided by NaHCO₃ and Na₂CO₂. For example bicarbonate and carbonate are present in a concentration of at least 30, 60, 90, 150, 210, 240 or at least 270 mM.

Preferably, the medium comprises any of the alkali metal chlorine salt or a combination thereof as described above, in a concentration in the range of 120 to 500 mM, preferably 200 to 350 mM. The concentration of the alkali metal chlorine salt in the medium is at least 120 mM, advantageously at least 200 mM, more advantageously at least 270 mM, yet more advantageously at least 350 mM, most advantageously at least 400 mM. The alkali metal chlorine salts are advantageously NaCl and/or KCl.

In a preferred embodiment according to the invention, the medium comprises:

200-350 mM, preferably 270 mM NaCl;

50-100 mM, preferably 75 mM NaHCO₂; and

50-100 mM, preferably 75 mM Na₂CO₃.

This particular composition of the medium surprisingly turns out to enable sufficient growth of algae, while at the same time a low level of precipitation is achieved.

Furthermore, the medium enables efficient carbon dioxide transfer.

Preferably the medium further comprises 0.005-0.02 mM Ca and 0.075-0.3 mM Mg and having a pH of 9.0-10.5.

In a preferred embodiment, the medium further comprises one or more suitable vitamins, in particular thiamine, biotin and/or vitamin B12.

In a preferred embodiment, the medium further comprises one or more suitable micronutrients, in particular manganese, zinc, copper, molybdenum and/or cobalt.

In a preferred embodiment, the medium further comprises one or more suitable macronutrients, in particular potassium, phosphorus and/or nitrogen.

The goal of the present invention achieved accordingly, to provide a method and a system for obtaining algal biomass by enhancing the effect of the cultivation pH on the maximal biomass yield and lipid productivity of N. oleoabundans during light-limited growth, specifically by maximizing the relation between the optimal values of obtained biomass density, productivity and yield on light energy at two different pH levels and at a fixed incident light intensity. In particular, without nutrient limitation, the maximal volumetric biomass productivity is approximately two-fold higher under reference conditions than under elevated pH and nitrogen depletion associated with high pH showed to be far more effective in promoting fatty acid accumulation than nitrogen depletion at reference pH. These results indicate the potential of producing high amounts of lipids by cultivating N. oleoabundans under the alkaline-saline conditions described in the present invention, possibly integrated in a two-stage process during which algal biomass is first grown under optimal conditions.

Further advantages, features and details of the invention are elucidated on basis of preferred embodiments thereof described in the Examples, wherein reference is made to the accompanying drawings, wherein:

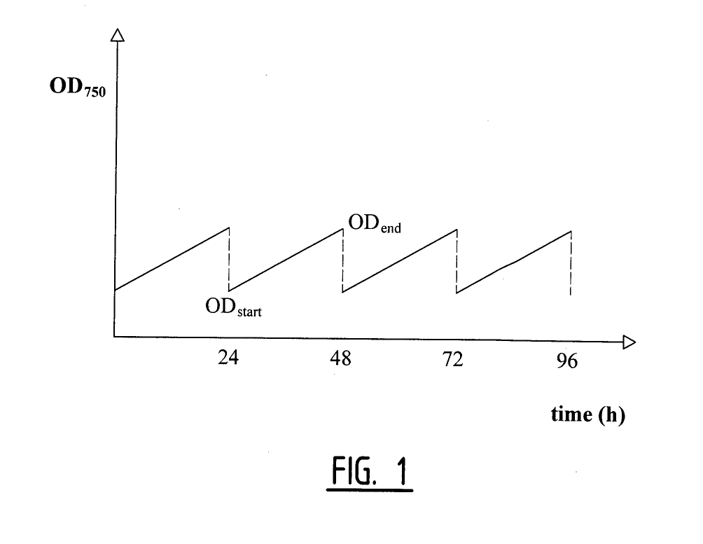

FIG. 1 shows a schematic representation of dilution steps in 24-well microtiter plates;

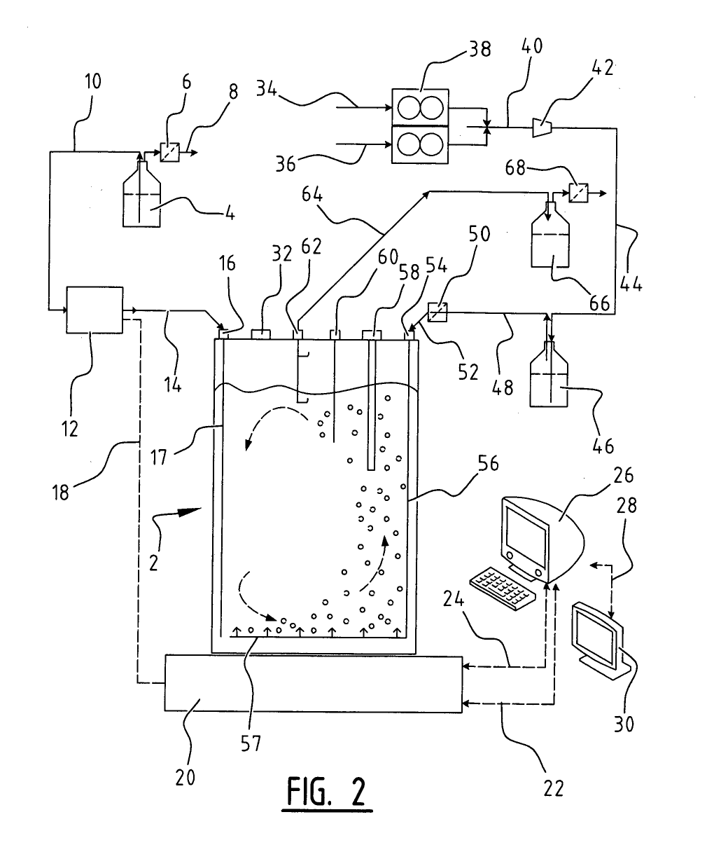

FIG. 2 shows a schematic representation of photobioreactor FMT150. Elements 4, 6, 8, 10, 12, 14, 18, 64, 66, 68 correspond to optional connections for operating in turbidostat mode;

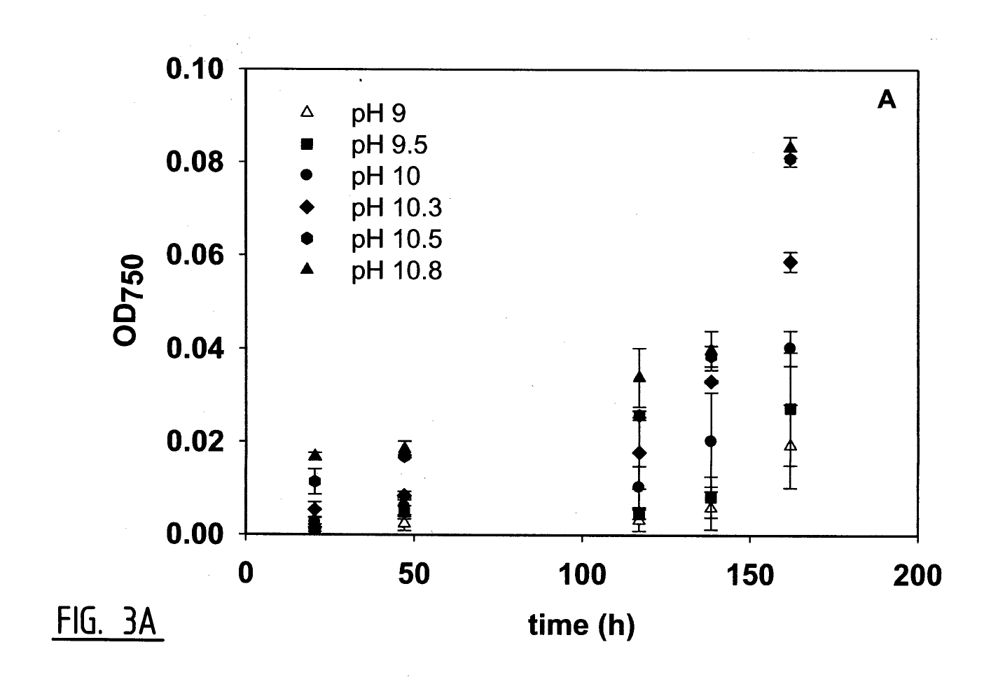

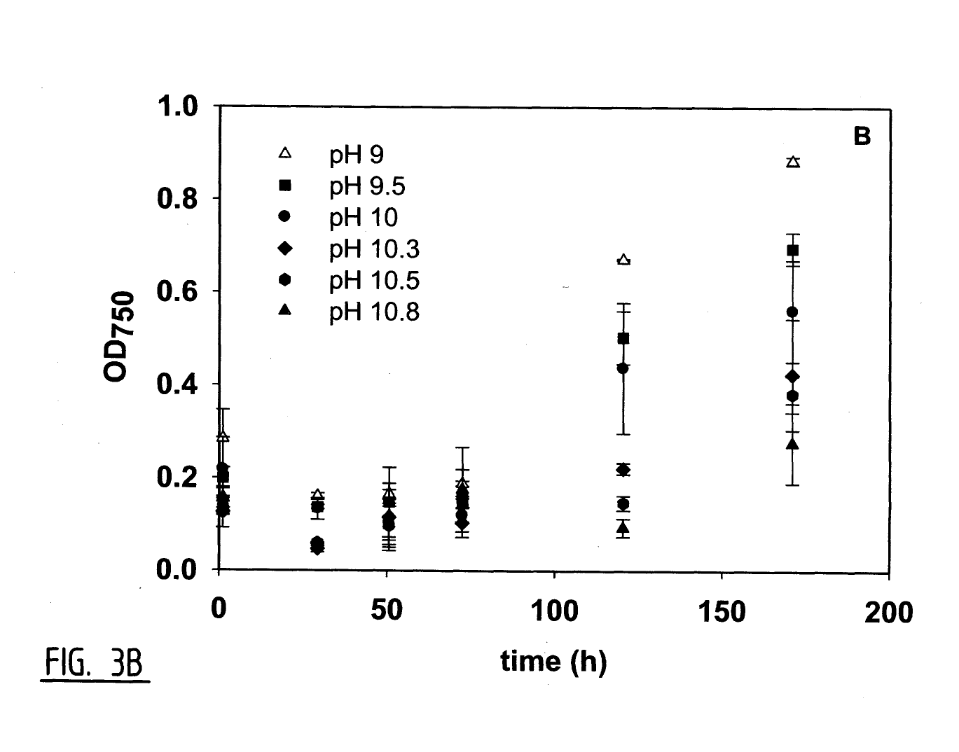

FIGS. 3A-B show the optical density at 750 nm (OD750) of (A) modified f/2 medium at different pH values where the increase in OD750 is a measure of medium precipitation and (B) N. oleoabundans in the modified f/2 medium at different pH values as a measure of growth. Lowest and highest pH values tested are represented by white and black triangles, respectively. Error bars represent the standard deviation from the average of the replicates;

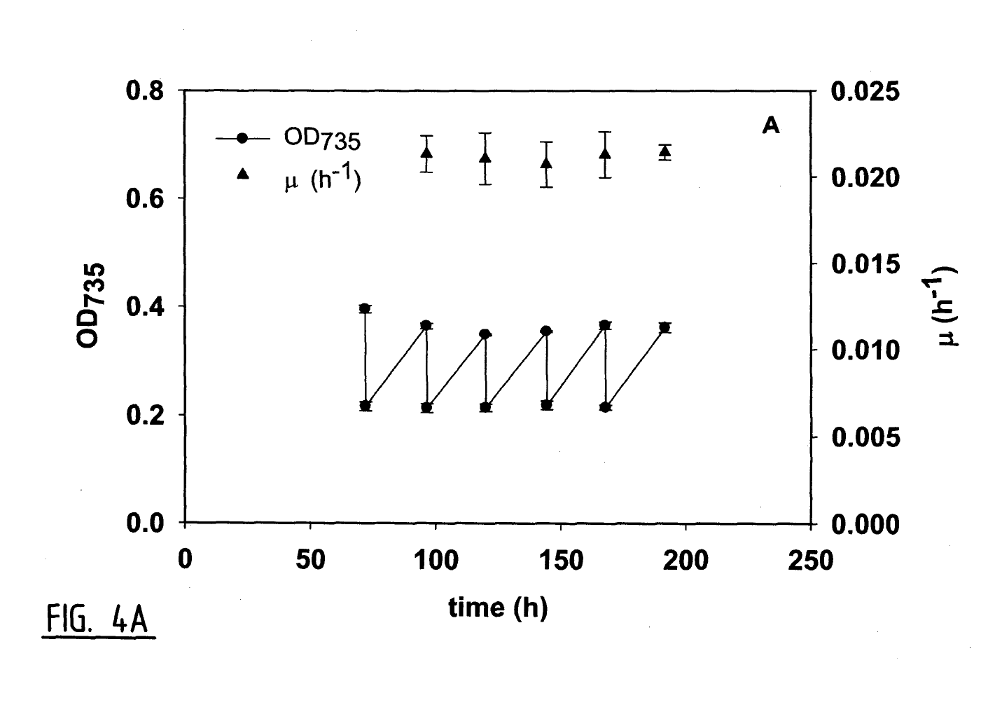

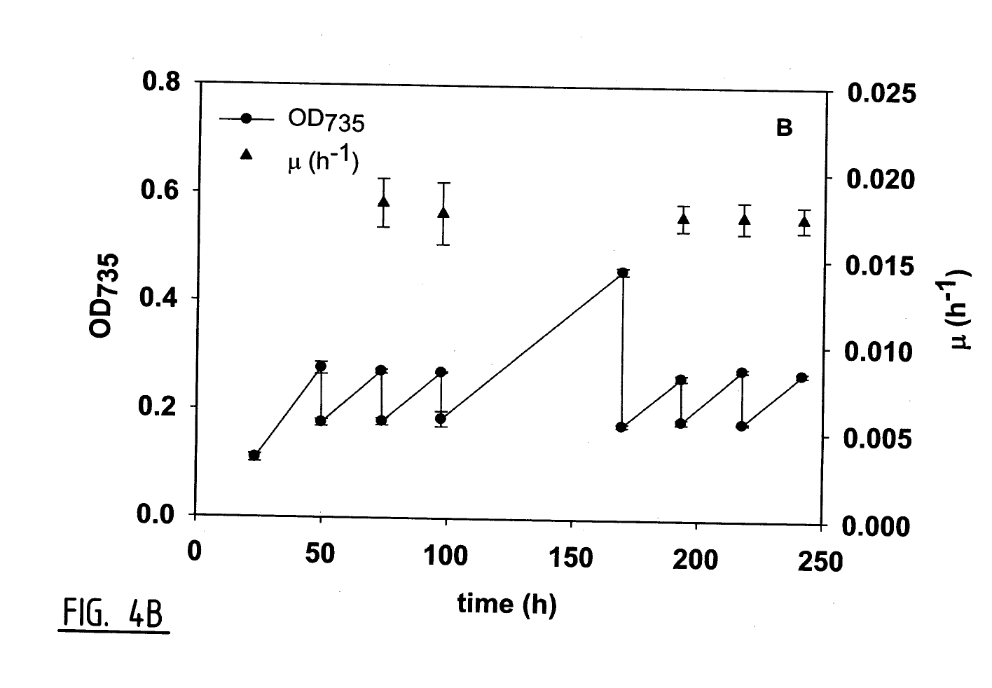

FIGS. 4A-B show the growth of N. oleoabundans in dilution series at (A) pH 8.1 and (B) pH 10.0. Growth rates determined 24 hours after each dilution step;

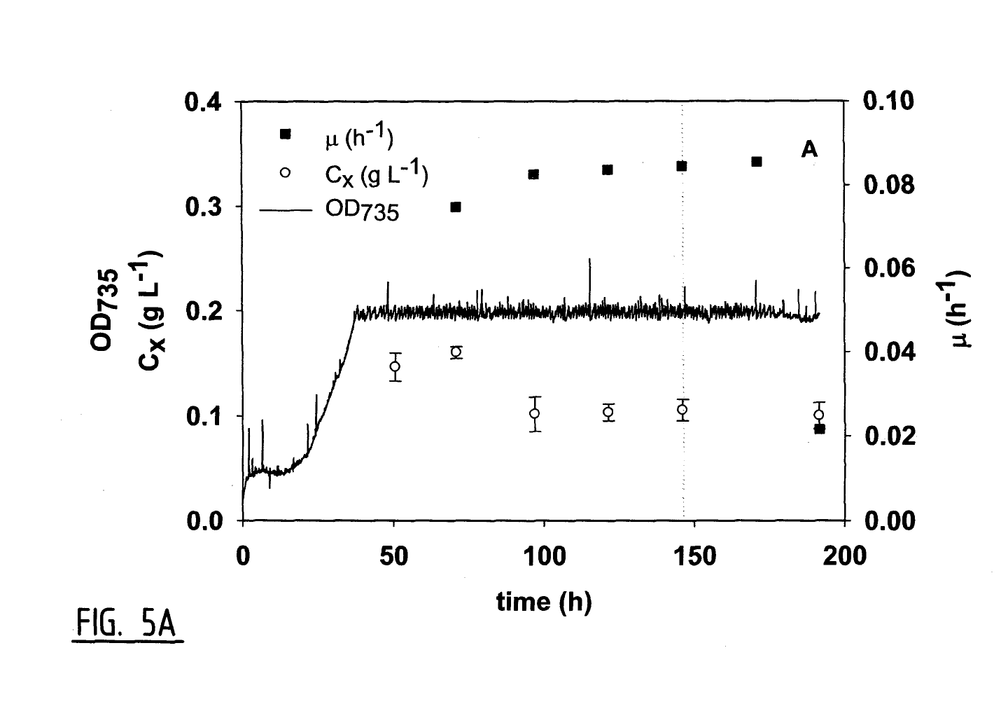

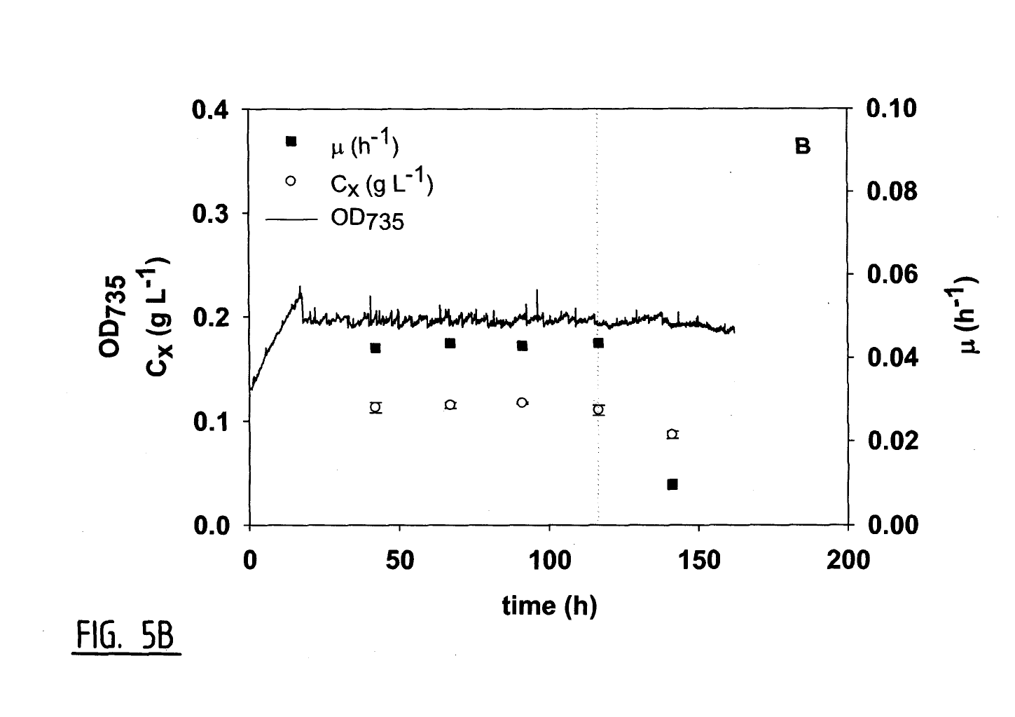

FIGS. 5A-B show the growth of N. oleoabundans in turbidostat cultivation at (A) pH 8.1 and (B) pH 10.0. The vertical dotted lines represent the point after what depletion of nitrogen from the medium was applied; and

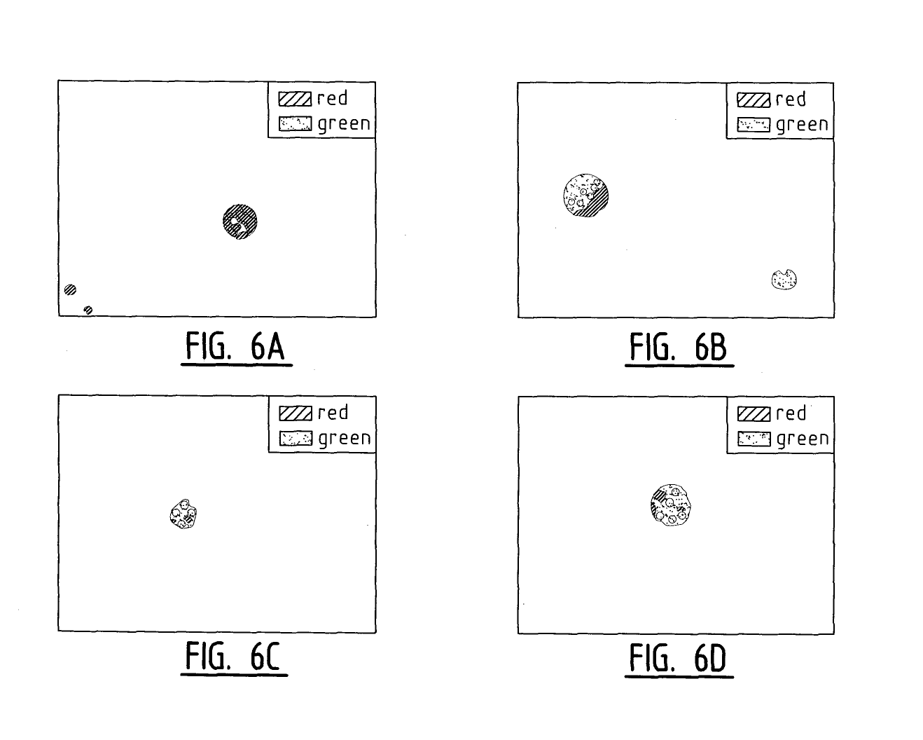

FIGS. 6A-D show pictures taken during the turbidostat experiment at pH 10.0. (A) When in N-replete medium, most part of the cells showed red fluorescence emitted by the chloroplasts. (B), (C) and (D) Green fluorescence emitted by lipid bodies in nitrogen starved cells. All images are 1000 times magnified.

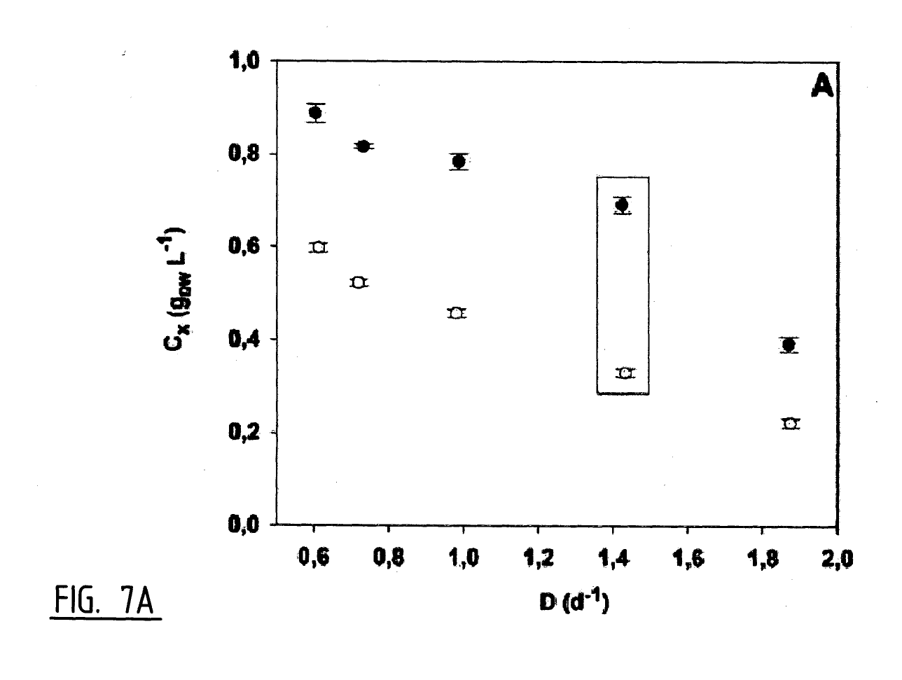

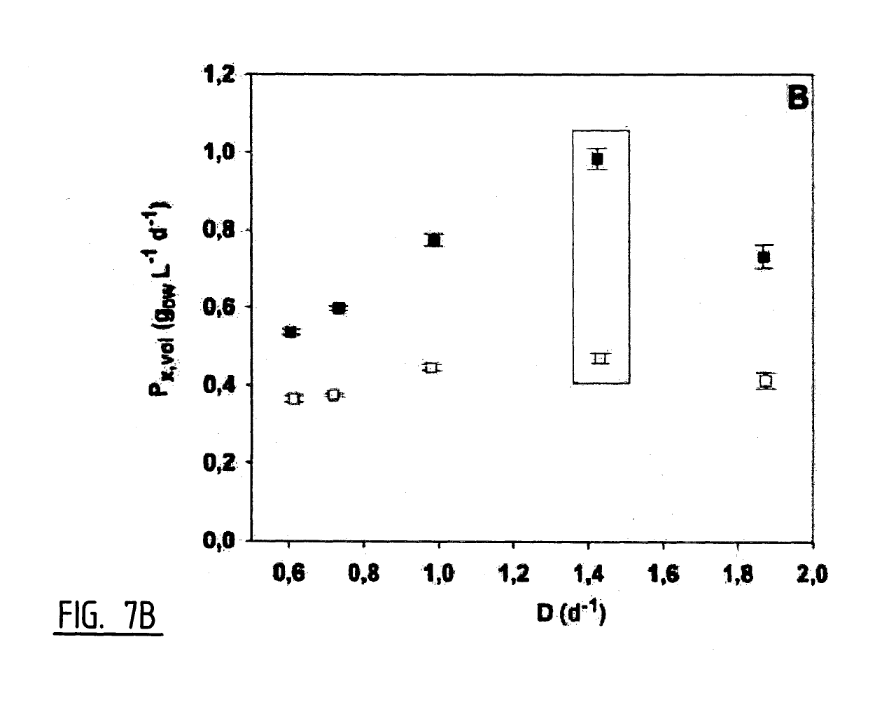

FIG. 7A-B shows the influence of dilution rate (D, d₋₁) on (A) biomass concentration (Cx, g_{DW} L₋₁) and (B) biomass productivity (P_{x,vol}, g_{DW} L₋₁ d₋₁) of N. oleoabundans at reference conditions (pH 8.2, closed symbols) and at higher pH (pH 10.0, open symbols). Values for maximal P_{x,vol} and corresponding optimal Cx are indicated by rectangles. Error bars represent the standard deviation from the average of the replicates.

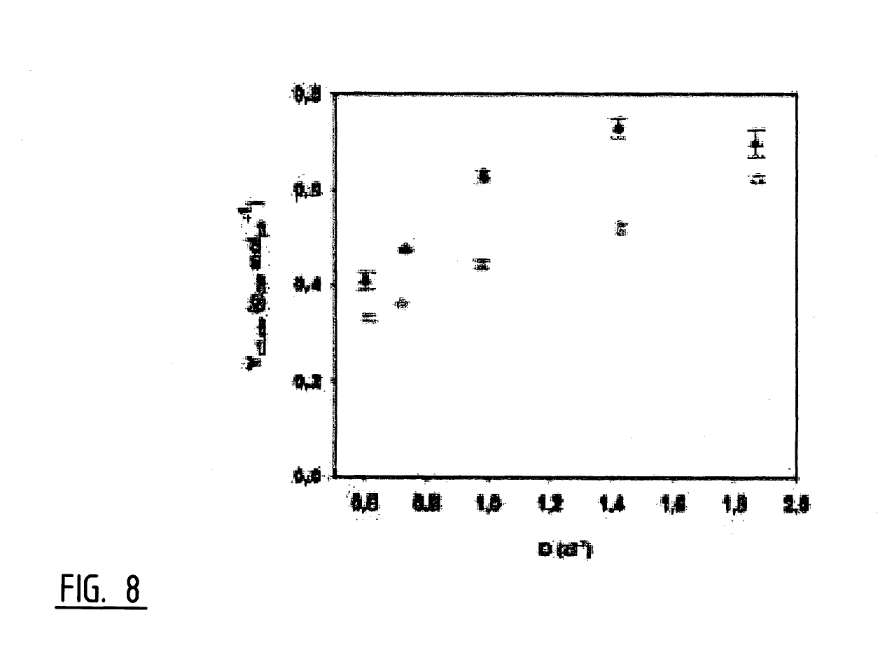

FIG. 8 show the influence of dilution rate (D, d₋₁) on the biomass yield on light energy (Y_{x/E}, g_{DW} molₚₕ₋₁) of N. oleoabundans at reference conditions (pH 8.2, closed symbols) and at higher pH (pH 10.0, open symbols). Error bars represent the standard deviation from the average of the replicates.

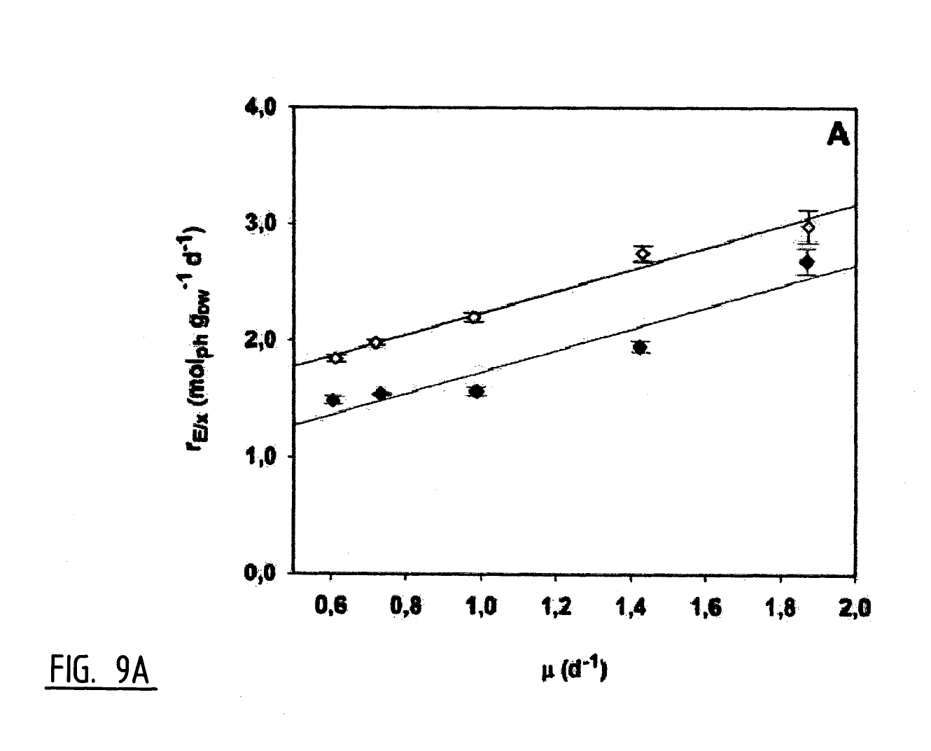

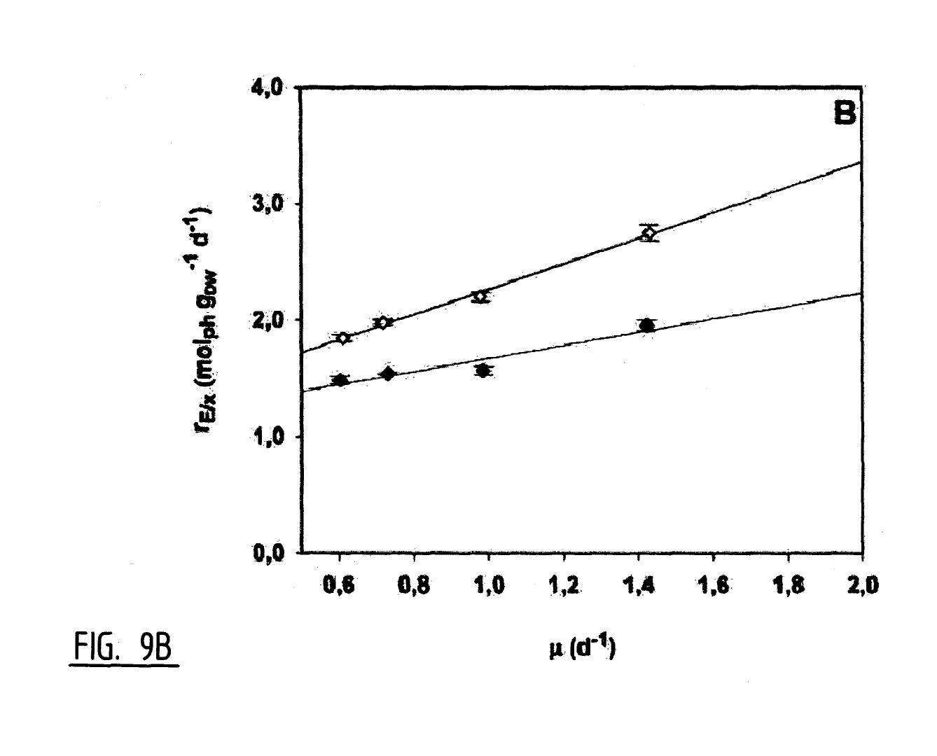

FIG. 9 show the influence of growth rate (μ=D, d₋₁) on the specific light absorption rate (r_{E/x}, molₚₕ g_{DW} d₋₁) of N. oleoabundans at reference conditions (pH 8.2, closed symbols) and at higher pH (pH 10.0, open symbols). (A) takes into account all data points measured during the experiments and (B) assumes that oversaturation takes place at D=1.9 d₋₁ and excludes the correspondent data points. Error bars represent the standard deviation from the average of the replicates.

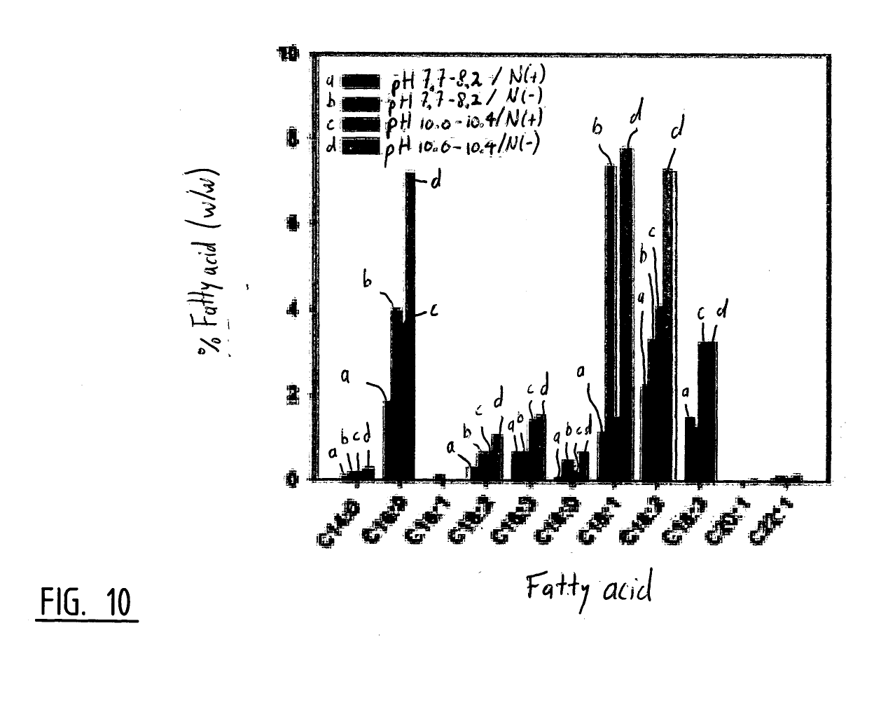

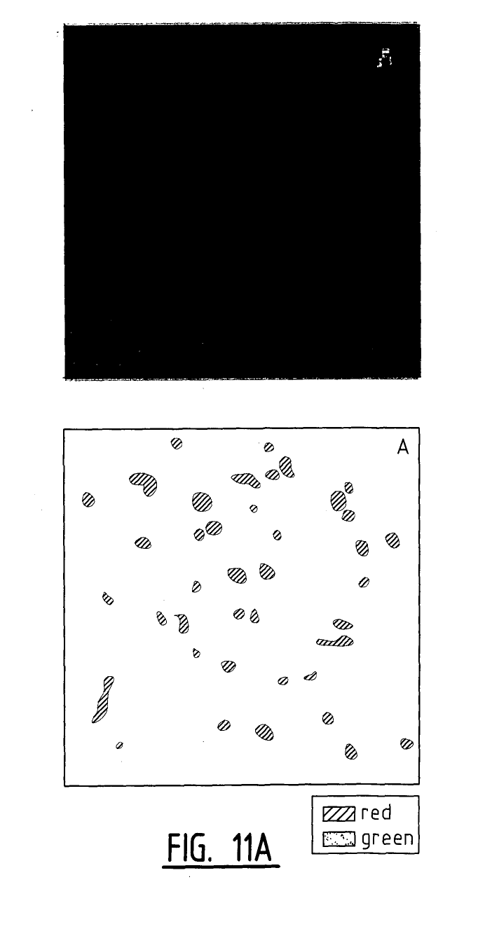

FIG. 10 show fatty acid composition (% w/w) of N. oleoabundans at the highest dilution rate applied (D=1.9 d₋₁). Results presented for reference conditions (pH 8.2) and for higher pH (pH 10.0) experiments, without and with nitrogen depletion (N(+) and N(−), respectively).

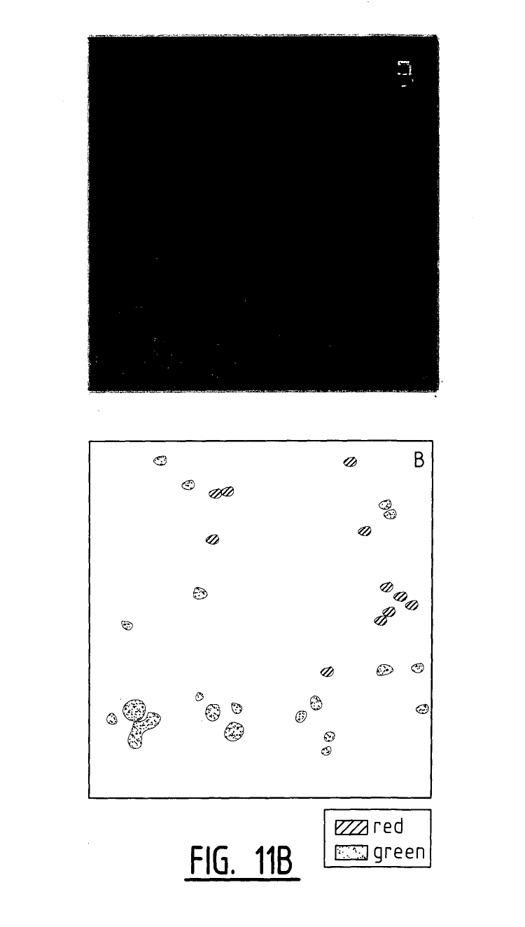

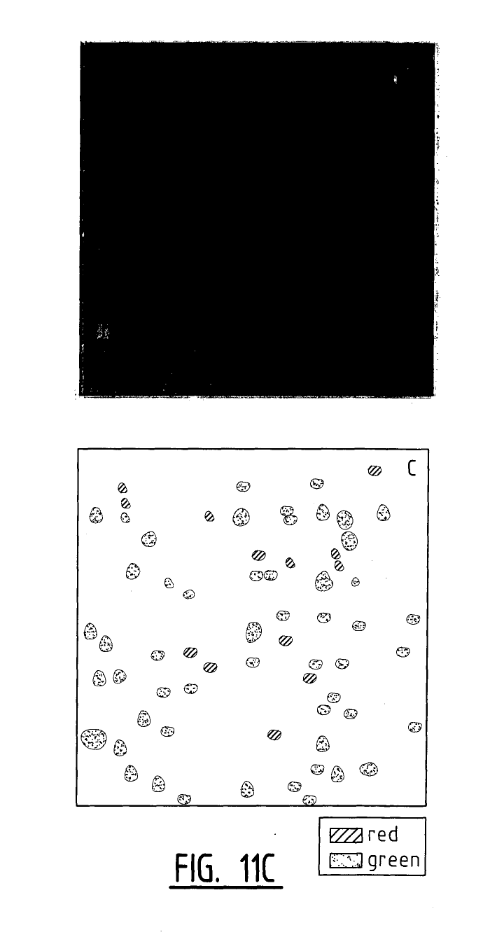

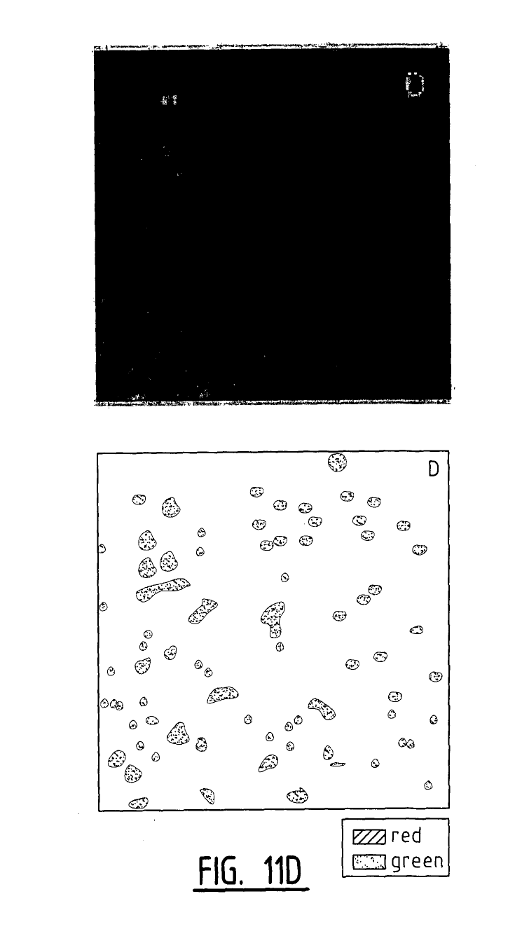

FIG. 11-Pictures taken during chemostat experiments at pH 10.0 after cell staining with Bodipy 505/515. (A) Red chlorophyll autofluorescence under N-replete conditions.

### EXAMPLES

### Example 1

### Materials and Methods

### Microalgal Strain

Experiments were performed in a lab-scale photobioreactor, and in microtiter plates to determine the specific growth rate of microalga of the species Neochloris oleoabundans under a range of alkalinities and salinities. Besides nutrient-replete growth, nitrogen deprivation was applied to induce lipid accumulation and to check whether this accumulation was affected by the alkalinity and the salinity of the growth medium. Moreover, the development of a stable medium under those conditions is discussed.

N. oleoabundans UTEX 1185 was obtained from the algae culture collection of the University of Texas, Austin, USA and pre-cultivated in 250 mL shake flasks containing 100 mL of growth medium. We placed them on a orbital shaker incubator (Innova 44, New Brunswick Scientific, New Jersey, USA) under a 2% CO₂ enriched headspace and a light intensity of 30 μmol photons m⁻²s⁻¹ provided by fluorescent lamps. Temperature was controlled at 25° C. and the pH was adjusted to 7.8.

### Medium Design and Macroalgal Growth

A modified f/2 medium was used for growth as described in table 1.

TABLE 1
Modified f/2 medium composition. X and Y are the concentrations
of magnesium and calcium in artificial seawater, respectively.
Values for these concentrations were determined in the
experiments, as described below.

| | |
|---|---|
| Compound | [mM] |

Macronutrients

| KNO₃ | 4.4 | |
|---|---|---|
| Super FK (P₂O₅) | 0.32 | |
| EDTA ferric | 29.28 | μM |
| sodium salt | | |

Vitamins

| | | |
|---|---|---|
| Thiamine | 200 | μL⁻¹ |
| Biotin | 1 | μL⁻¹ |
| B₁₂ | 1 | μL⁻¹ |

Micronutrients

| | |
|---|---|
| EDTA-MnNa₂ | 1.5E−02 |
| EDTA-ZnNa₂ | 2.0E−03 |
| EDTA-CuNa₂ | 7.0E−03 |
| Na₂MoO₄•2H₂O | 7.7E−04 |
| CoCl₂•6H₂O | 3.7E−04 |

Artificial seawater

| | |
|---|---|
| NaCl | 420.0 |
| MgCl₂•6H₂O | X |
| CaCl₂•2H₂O | Y |
| Na₂SO₄ | 22.53 |

Non-biological precipitation experiments were performed in 24-well microtiter plates (Falcon 3047, BD Biosciences, California, USA), by manipulating the cultivation medium composition in order to test its stability at elevated pH. The plates were fixed inside the orbital shaker incubator rotated at 150 rpm with an orbit of 2.54 cm. Wells with demineralised water were taken as blanks for the measurement of optical density (OD). In a total volume of 1.5 mL per well, different Mg²⁺/Ca²⁺ combinations in f/2 medium were tested. The ratio between both elements was either kept constant (1:13), or varied with a constant minimal calcium concentration (0.01 mM). Phosphate and nitrate were kept in a ratio of 1:14 at levels sufficient to sustain 1 g L⁻¹ of biomass. The phosphate source in this modified medium was a potassium polyphosphate solution insensitive to high pH levels (Super FK, YARA, Vlaardingen, The Netherlands). Six different pH values were tested in duplicate for each combination: 9.0, 9.5, 10.0, 10.3, 10.5 and 10.8. The desired pH was obtained by buffering the wells with mixtures of sodium bicarbonate (NaHCO₃) and disodium carbonate (Na₂CO₃) in a total concentration of 300 mM, as described in table 2.

TABLE 2
Molar concentrations of NaHCO3 and Na2CO3 in
the buffering mixtures, for each pH tested.

| pH | NaHCO₃ [mM] | Na₂CO₃ [mM] |
|---|---|---|
| 9.0 | 270 | 30 |
| 9.5 | 210 | 90 |
| 10.0 | 150 | 150 |
| 10.3 | 90 | 210 |
| 10.5 | 60 | 240 |
| 10.8 | 30 | 270 |

The stability of the medium was assessed by following the optical density at 750 nm (OD750) in time using a multilabel plate reader (VICTOR™ X3, PerkinElmer, Massachusetts, USA). The light scattered by the insoluble particles (i.e. salt crystals) in suspension determines the optical density at 750 nm and can be used as a measure of the formation of those particles.

The growth of N. oleoabundans was studied in this modified medium, under the same conditions, except for the supply of light and carbon dioxide. Light was supplied by fluorescent light tubes at an intensity of 30 μmol photons m⁻²s⁻¹ and CO₂ by applying a headspace with 4% CO₂-enriched air.

In these experiments, 75 μL from 2 weeks old shake flask cultures were used as inoculum in a total of 1.5 mL per well. Wells filled with solely medium were used as controls. The optical density at 750 nm was measured daily to follow biomass growth.

Growth Assessments in 24-Well Microtiter Plates

The influence of a range of alkalinities and salinities on growth was also studied in 24-well microtiter plates. The wells were filled with 1.350 mL of the modified f/2 medium and 150 μL of inoculum from 2 weeks old shake flasks cultures. Wells filled with solely 1.5 mL of medium were used as blanks for the measurement of optical density (OD). The plates were placed in a chamber fixed inside the orbital shaker incubator, rotated at 150 rpm with an orbit of 2.54 cm. Continuous light was supplied by fluorescent lamps at an intensity of 55 μmol photons m⁻²s⁻¹. The chamber's headspace was continuously flushed with humidified and CO₂-enriched air. Both gas flows were set by mass flow controllers (Bronkhorst Nederland B.V., Veenendaal, The Netherlands). Microalgae growth was assessed at 30° C. and four pH values: 8.1, 9.0, 10.0 and 10.5. The pH was set by buffering the growth medium with mixtures of NaHCO₃ and Na₂CO₃, in total concentrations of 300 mM and 150 mM (table 3) and adjusting the CO₂ fraction in the headspace.

TABLE 3
Molar concentrations of NaHCO3 and Na2CO3 in
the buffering mixtures used in total concentrations
of 300 and 150, for each pH tested.

| | Total [mM] | | | |
|---|---|---|---|---|
| | 300 | | 150 | |
| pH | NaHCO₃ [mM] | Na₂CO₃ [mM] | NaHCO₃ [mM] | Na₂CO₃ [mM] |
| 9.0 | 270 | 30 | 135 | 15 |
| 10.0 | 150 | 150 | 75 | 75 |
| 10.5 | 60 | 240 | 30 | 120 |

By varying the concentration of sodium chloride (NaCl), also the algae's salt tolerance was checked. In four sets of experiments, different concentrations of NaCl in the medium were combined with the NaHCO₃ and Na₂CO₃ mixtures (table 4).

TABLE 4
Sodium chloride combinations with the NaHCO₃ and Na₂
CO₃ buffering mixtures, used in each set of experiments.

| Set | NaCl [mM] | NaHCO₃ + Na₂CO₃ [mM] |
|---|---|---|
| 1 | 420 | 300 |
| 2 | — | 300 |
| 3 | 120 | 300 |
| 4 | 270 | 150 |

This was done at each pH value, except for the reference at pH 8.1, where only f/2 medium with 12 mM NaHCO₃ was used. All sets of experiments were performed in four replicates to check reproducibility.

Optical density at 750 nm was measured daily using a multilabel plate reader (VICTOR™ X3, PerkinElmer, Massachusetts, USA). After 24 hours of inoculation, the suspensions with known initial volumes (Vs) and OD750 (ODₛₜₐᵣₜ) were diluted with a certain volume of medium (Vm) in such a way that after another 24 hours of growth the same OD750 (OD_{end}) was reached. Specific growth rates (μ) were then calculated for all the replicates according to equation A:

$$μ = \frac{ln \{ {OD}_{end} / [ \frac{{OD}_{start} \times {V}_{s}}{{V}_{s} + {V}_{m}} ] \}}{24} \qquad ( equation A )$$

The average μ and corresponding standard deviations were determined and used to estimate growth. The dilution series were done every 24 hours, until a constant specific growth rate was observed, as schematically represented in FIG. 1, after which the cells were allowed to grow until the stationary phase.

### Growth Assessments in a Flat Panel Photobioreactor

### Photobioreactor Set-Up

The microalgae were also grown in a flat panel photobioreactor (FMT150, Photon Systems Instruments, Brno, Czech Republic) with a detachable rectangular cuvette 20 cm high, 10 cm wide and 2.5 cm of light path.

Medium container 4 is connected via filter 6 to outlet 8 and via line 10 with peristaltic pump 12. Pump 12 is connected via line 14 with medium inlet 16. Via line 17 the medium is directed towards the bottom of reactor 2. Pump 12 is connected via control signal line 18 to control unit 20, which is connected via data visualisation line 22 and data collection line 24 to local computer processing unit 26. Processing unit 26 is optionally connected to remote PC 30 via line 28.

Photobioreactor 2 comprises an optional sensor 32 for instance for measuring optical density. Preferably the sensor is connected to control unit 20.

Air inlet 34 and CO₂ inlet 36 are connected via mass flow controllers 38 and line 40 to bubble interruption valve 42, which is connected via lines 44 to gas humidification bottle 46. Line 48 connects the gas humidification bottle 46 via filter 50 and line 52 to gas inlet 54 of the reactor 2. Gas inlet 54 is connected via line 56 to sparger 57 which releases the gas at the bottom of reactor 2.

Reactor 2 further comprises a pH sensor 58, which is preferably connected to control unit 20. Furthermore, a sample outlet 60 and a gas/liquid outlet 62 are provided. Gas/liquid outlet 62 is connected via line 64 to overflow unit 66 which is connected to filter 68.

The photobioreactor (PBR) 2 (FIG. 2) was equipped with temperature control and a light source composed of red emitting diodes, LEDs (λmax=627 nm). A built-in dual-wavelength densitometer 32 allowed us to monitor the culture growth and the chlorophyll absorption through the integrated measurement of optical density at 735 nm and 680 nm, using the PBR itself as cuvette (2.5 cm optical path). In turbidostat mode the PBR also controlled a peristaltic pump 12 in order to maintain a constant OD735 by the addition of fresh medium and concomitant harvesting of surplus culture.

The incident light flux was measured at 15 different places equally distributed over the PBR's light-exposed surface with a LI250 PAR 2π quantum sensor (Li-COR®, Lincoln, USA). The average light at the surface was set to 300 μmol photons m⁻²s⁻¹ and the temperature was maintained at 30° C. Mixing was performed by continuous sparging at the bottom of the cuvette with a filtered CO₂-enriched air stream, saturated with water before entering the vessel. The flows of air and CO₂ were set by mass flow controllers (Bronkhorst Nederland B.V., Veenendaal, The Netherlands) to a total of 650 ml min⁻¹. The flow of CO₂ in the gas stream was adjusted according to the pH required in the study.

### Microalgae Growth in Turbidostat Cultivation and Lipid Accumulation Under Nitrogen Depletion

The influence of the pH on growth was studied in turbidostat mode at pH 8.1 and pH 10.0. At both pH the algae were deprived of nitrogen at the end of the experiments to induce the accumulation of lipids (oil bodies). The concentration of NaCl in the medium was 419 mM at pH 8.1 and 270 mM at pH 10.0. The pH was determined by the CO₂ supplied through the gas stream and by the addition of NaHCO₃ and Na₂CO₃ to the medium. At pH 8.1, 12 mM NaHCO₃ was in equilibrium with 1% v/v CO₂. At pH 10.0, a mixture of 75 mM NaHCO₃ and 75 mM Na₂CO₃ was necessary while gassing with only 0.1% v/v CO₂.

The working volume of the photobioreactor (FIG. 2) was 385 mL composed of 355 mL of filtered medium and 30 mL inoculum from a 2-week old shake flask culture. Growth started batch wise in the modified f/2 medium (table 1) with optimal Ca²⁺ and Mg²⁺ salt concentrations (X and Y, see results) and the amounts of NaHCO₃ and Na₂CO₃ given above. An OD735 of 0.2 was defined as the set-point (e.g. FIG. 4). At this point the turbidostat mode was activated and the suspension's optical density was kept constant, by dilution with fresh medium each time values above 0.2 were measured. This allowed determining p under steady-state, which was equivalent to the dilution rate (D). Additionally, the correspondent biomass concentrations (Cx) were determined by dry weight measurements.

Nitrogen depletion was applied when the growth rate was constant in nitrogen-replete conditions, by replacing the medium with N-free medium resulting in a gradual but complete depletion of nitrogen.

Biomass Determination

Triplicate samples of ten grams of the algae suspension were filtrated over pre-dried and pre-weighted Whatman GF/F glass fiber filters (Ø 55 mm, nominal pore size 0.7 μm). The wet filters were then dried at 95° C. for 24 hours, placed in a dessicator to cool down for 2 additional hours and weighted again. The dry weight was determined as the difference between the weights of the dried filters containing the samples and the dried empty filters.

Visualization of Intracellular Neutral Lipid Bodies

The accumulation of intracellular lipid bodies by the algae was checked by fluorescence microscopy. The lipophilic fluorescent dye BODIPY 505/515 (4,4-difluoro-1,3,5,7-tetramethyl-4-bora-3a,4a-diaza-sindacene; Invitrogen Molecular Probes, Carlsbad, Calif.) was used for staining the cells, according to Cooper et al. (2010). A 40 μM BODIPY 505/515 stock solution was prepared by dissolving the dye in 0.2% (v/v) of anhydrous dimethyl sulfoxide (DMSO), used as a vehicle to facilitate the permeation of the dye into the cells. Aliquots of this stock solution were added directly to the algal cell suspensions in order to have a final dye concentration of 1 μg mL¹ and 0.02% DMSO. After 5 minutes of contact at 25° C., in the dark with agitation, slides were prepared for observation under an inverted fluorescent microscope (Leica DMI6000, Leica Microsystems B.V., Rijswijk, The Netherlands). Excitation was done under a blue excitation light through a band-pass filter at 450-490 nm. Emission wavelengths were imaged through a long-pass filter of 515 nm. BODIPY 505/515 has a green emission spectrum which makes the stained lipid bodies distinct from the endogenous red autofluorescence emitted by the chloroplasts under the same blue excitation light.

Fatty Acids Quantification

Duplicate samples of the algae suspension with a known volume were centrifuged and the wet pellets were frozen and stored at −80° C. Before the fatty acids extraction itself, those pellets were resuspended and transferred to bead beating tubes. Those tubes were lyophilized overnight and the exact amount of biomass was determined. 1 mL of a 2:2.5 mixture of chloroform and Methanol containing 42 μg mL¹ of Tripentadecanoin (C15:0 TAG, Sigma-Aldrich T4257, as internal standard) was used to dissolve the dried biomass. To disrupt the cells, the bead beating tubes were placed in a bead beater for 40 minutes. The liquid samples were then transferred to 10 mL Pyrex tubes with Teflon coated screw caps. Additional 3 mL of the same mixture were used to rinse the bead beating tubes to recover the whole biomass. After proper homogenization, the tubes were placed in a water bath for sonication during 10 min. 2.5 mL of 50 mM Tris-buffer (containing 1M NaCl, pH=7.5) were added to separate the polar from the apolar phase. The tubes were then centrifuged for 5 minutes at 2500 rpm and 15° C. The phase separation bottom layers (first chloroform fractions) were transferred to new tubes while cell debris and the polar phases were re-extracted twice with 1 mL of chloroform. The separation of the pooled chloroform from the algal material was done by evaporation under pure nitrogen for around 45 minutes (or until all the chloroform was gone). 3 mL of 5% (v/v) sulfuric acid in methanol was added to the tubes to convert the fatty acids into fatty acid methyl esters (FAMEs). The samples were then incubated in a block heater for 3 hours at 70° C. After cooling down the samples to room temperature, 3 mL of hexane and 3 mL of demineralized water were added and the tubes were centrifuged for 5 minutes at 2500 rpm and 15° C. Aliquots of around 2 mL where collected from the phase separation upper layer (hexane and fatty acids) to new tubes. 2 mL of demineralized water were added to wash the hexane during a last step of centrifugation. The hexane phases containing the fatty acids were analyzed by gas chromatography (GC) using a HP 6890 (Hewlett Packard Inc., Supelco Nucol™ col. 355 33-03A, 30m×530 μm×1.0 μm). The FAMEs were identified by comparison of their retention times with those of standard fatty acids (Sigma Aldrich, Missouri, USA).

Quantification of the identified lipids was done according to the biomass dry weight and by comparing their respective peak areas. Amounts were corrected for the response factors of each fatty acid.

### Results and Discussion

Growth of N. oleoabundans in a Modified f/2 Medium

The preparation of cultivation media with high concentrations of salts and high pH needs careful attention regarding the issue of precipitation. Compounds which precipitate are no longer available for microalgal growth. For instance, the binding capacity of calcium to phosphate is pH dependent, increasing at elevated values.

A chemical equilibrium model for the calculation of saturation indices in aqueous solutions was the base for developing a medium with low levels of precipitation. A simulation showed for example high formation of a calcium phosphate complex, hydroxyapatite, for our modified f/2 medium with a pH of 10.5 containing Ca²⁺ and PO₄³⁻ in concentrations of 3.61 mM and 5 mM, respectively. One way to reduce the presence of hydroxyapatite is to decrease the concentrations of Ca₂₊ and/or PO₄³⁻.

The elemental composition of Chlorella vulgaris, was used as a basis for determining the concentrations of nitrogen and phosphate necessary to produce 1 g L⁻¹ of biomass, defined to be sufficient for this study. It was determined that 4.4 mM NO³⁻ and 0.32 mM PO₄³⁻ need to be present in solution to reach such biomass concentration.

Non-biological experiments showed that Ca²⁺ in a concentration of 0.01 mM leads to a medium with very low complexation levels at high pH values. Besides calcium, also magnesium can complex and precipitate at high pH, though at a lesser degree. Moreover, the amount of magnesium needed by the cells seems to be higher than calcium. The best ratio we obtained between Ca²⁺ and Mg²⁺ was 1:15, the latter being in a concentration of 0.15 mM.

According to the present invention, the medium is based on f/2 medium with 0.01 mM CaCl₂ (X), 0.15 mM MgCl₂ (Y), 4.4 mM KNO₃ and 0.32 mM ‘P’ (from the polyphosphate). In FIG. 3A the optical density shows the level of precipitation at different pH values. In the first 100 hours the OD hardly increases. Only at elevated pH (>10) and after extended time (150 hours) limited precipitation occurs. For continuous microalgae production this should not cause any problems since the rate of consumption will be faster than the rate of precipitation. Moreover, continuous consumption will result in even lower concentrations of Ca²⁺, Mg²⁺ and PO₄³⁻, further reducing the risk of precipitation.

When cultivating N. oleoabundans in the modified medium a decrease in growth with the increased pH was observed (FIG. 3B). However, growth was apparently not limited by the low concentrations of Ca²⁺ and Mg²⁺ and the cells could reach a maximal OD750 of about 0.9 at pH 9.0 and of almost 0.6 at pH 10.0.

The values of the graphs of FIG. 3 are listed below in table 5A-B.

TABLE 5A
Optical density at 750 nm (OD₇₅₀) of modified
medium at different pH values where the increase in OD₇₅₀ is
a measure of medium precipitation.

| | pH 9 | | pH 9.5 | | pH 10 | |
|---|---|---|---|---|---|---|
| time | | std | | std | | std |
| (h) | OD750 | error | OD750 | error | OD750 | error |
| 20.518 | 1.83E−03 | 3.17E−04 | 1.04E−03 | 4.24E−04 | 2.65E−03 | 1.05E−03 |
| 47.085 | 2.37E−03 | 1.47E−03 | 4.81E−03 | 1.49E−03 | 6.11E−03 | 1.87E−03 |
| 116.96 | 3.19E−03 | 2.29E−03 | 4.66E−03 | 1.17E−03 | 0.0104 | 4.39E−03 |
| 138.37 | 5.85E−03 | 4.66E−03 | 8.24E−03 | 4.49E−03 | 0.02 | 0.0105 |
| 161.95 | 0.0191 | 8.86E−03 | 0.0271 | 0.0121 | 0.04 | 3.78E−03 |
| | pH 10.3 | | pH 10.5 | | pH 10.8 | |
| | | std | | std | | std |
| time (h) | OD750 | error | OD750 | error | OD750 | error |
| 20.5175 | 5.36E−03 | 1.72E−03 | 0.0114 | 2.71E−03 | 0.0168 | 8.20E−04 |
| 47.0847 | 8.43E−03 | 8.96E−04 | 0.0168 | 3.22E−04 | 0.0184 | 1.69E−03 |
| 116.955 | 0.0176 | 7.60E−03 | 0.0256 | 9.60E−04 | 0.0337 | 6.30E−03 |
| 138.367 | 0.0329 | 3.00E−04 | 0.0383 | 2.21E−03 | 0.0395 | 4.20E−03 |
| 161.952 | 0.0585 | 2.13E−03 | 0.0808 | 1.68E−03 | 0.0832 | 2.24E−03 |

TABLE 5B
Optical density at 750 nm (OD750) of N. oleoabundans
in the modified medium at different pH values
as a measure of growth.

| (B) | pH 9 | | pH 9.5 | | pH 10 | |
|---|---|---|---|---|---|---|
| time | | std | | std | | std |
| (h) | OD750 | error | OD750 | error | OD750 | error |
| 1.0897 | 0.2838 | 0.0622 | 0.1993 | 0.0228 | 0.2172 | 0.0689 |
| 29.259 | 0.1607 | 6.90E−03 | 0.1374 | 4.64E−03 | 0.134 | 0.0231 |
| 50.595 | 0.1653 | 9.27E−03 | 0.1478 | 0.0746 | 0.1056 | 0.0487 |
| 72.383 | 0.1875 | 5.52E−03 | 0.1485 | 0.0126 | 0.1202 | 0.0353 |
| 120.38 | 0.6704 | 1.95E−03 | 0.5025 | 0.0572 | 0.4364 | 0.141 |
| 170.9 | 0.883 | 9.03E−03 | 0.6942 | 0.0346 | 0.5597 | 0.1095 |
| | pH 10.3 | | pH 10.5 | | pH 10.8 | |
| (B) | | std | | std | | std |
| time (h) | OD750 | error | OD750 | error | OD750 | error |
| 1.0897 | 0.1265 | 0.0338 | 0.1378 | 0.018 | 0.1576 | 0.0226 |
| 29.2589 | 0.0472 | 6.66E−03 | 0.0608 | 9.24E−04 | 0.0537 | 4.29E−03 |
| 50.5953 | 0.1162 | 0.0721 | 0.0981 | 0.0468 | 0.1027 | 0.0359 |
| 72.3825 | 0.1037 | 0.0307 | 0.1624 | 0.0556 | 0.1756 | 0.0904 |
| 120.38 | 0.2197 | 0.0122 | 0.146 | 0.0156 | 0.0941 | 0.0198 |
| 170.895 | 0.4223 | 0.1202 | 0.3798 | 0.0391 | 0.274 | 0.0862 |

### Example 2

### Influence of pH and Salt Concentration on Specific Growth Rate

### Microalgae Growth in 24-Well Microliter Plates

The methods for producing the microalgal strain and growth medium, as well as the photobioreactor are such as described in Example 1.

For all the pH values tested, no growth was observed with Set 1 (highest total salt concentration), suggesting that N. oleoabundans cannot stand such saline circumstances. At higher pH levels with less HCO³⁻/CO₃²⁻ (total 150 mM), the combination with more NaCl (270 mM/Set 4) seems to be better for the algae than less NaCl (absent/Set 2 and 120 mM/Set 3) combined with more HCO³⁻/CO₃²⁻ (total 300 mM). A medium with 270 mM NaCl and 150 mM HCO³⁻/CO₃²⁻ is therefore at this moment considered the optimal formulation capable of supporting growth at pH 10.0. Very poor growth was observed at pH 10.5, for all sets of experiments. Growth rates were not determined for this value.

An average growth rate of 0.0212 h⁻¹ was found for the reference experiment at pH 8.1 (FIG. 5A), being the highest amongst all the experiments. However, this value was not considerably different for the ones found at pH 10.0, where the specific growth rate reached 0.0177 h⁻¹ (FIG. 5B).

A correlation between the pH and the salt concentration seems evident and both have an impact on microalgal growth. Although the specific growth rates are all relatively low, due to light limitation inside the incubators (˜55 μmol photons m⁻²s⁻¹), they give a good insight on which should be the best conditions to grow N. oleoabundans at high pH: a marine medium with 270 mM NaCl, 75 mM NaHCO₂ and 75 mM Na₂CO₃.

### Example 3

### Microalgae Growth in Turbidostat Cultivation and Lipid Accumulation Under Nitrogen Depletion

The methods for producing the microalgal strain and growth medium, as well as the photobioreactor are such as described in Example 1.

To estimate the growth under high pH and salt concentration under controlled conditions in a continuous system, we performed two turbidostat experiments, at pH 8.1 and pH 10.0. In addition, the light intensity applied was of saturating nature (300 μmol photons m⁻²s⁻¹) and allowed to assess the specific growth rate without any light limitation. The total salt concentration was 420 mM according to the study performed in the microtiter plates. The respective biomass concentrations (Cx) and dilution rates (D) were determined under steady-state. In steady state the specific rate of cell growth equals the rate of dilution. Growth rates of 0.08 h⁻¹ and 0.04 h⁻¹ were found at pH 8.1 and pH 10.0 (FIGS. 6A and 6B, respectively). Cx was constant under steady-state, being about 0.1 g L⁻¹ in both experiments. Although the growth rate we obtained at pH 10.0 was twice as small as that at pH 8.1, it is still comparable with previous values obtained in the prior art in freshwater medium (bold basal medium) at pH 7.5 and 25° C.

Nitrogen starvation is known to be a strong inducer of lipid accumulation. After reaching a constant growth rate, the cultivation medium was replaced by nitrogen-free medium. This resulted on a sharp decrease in μ, to 0.02 h⁻¹ at pH 8.1 and 0.01 h⁻¹ at pH 10.0. This decrease is obviously caused by the absence of nitrogen in the medium which was already reported to be a growth limiting factor.

Cells growing in the photobioreactor at pH 8.1 and 10.0 were also analyzed for their total fatty acid content and composition, both under nitrogen-replete conditions and after 1 day of nitrogen depletion. Table 6 shows the microalgal content in each of the identified fatty acid, as well as their relative percentage of the total fatty acid pool.

TABLE 6
Fatty acid content (% w/w) of Neochloris
oleoabundans grown at pH 8.1 and pH 10.0 in turbidostat
experiments, without nitrogen limitation (N+) and with
nitrogen depletion (N−). The correspondent relative
compositions (% rel of the total) are also shown.
Content (% w/w)

| | pH | | | | | | pH | |
|---|---|---|---|---|---|---|---|---|
| Fatty | 8.1 | | pH 8.1 | | pH 10.0 | | 10.0 | |
| acid | N+ | %ᵣₑₗ | N− | %ᵣₑₗ | N+ | %ᵣₑₗ | N− | %ᵣₑₗ |
| C16:0 | 2.41 | 31.1 | 3.92 | 29.6 | 2.74 | 23.8 | 7.79 | 22.3 |
| C16:1 | 0.22 | 2.8 | — | — | 0.31 | 2.7 | 0.14 | 0.4 |
| C18:0 | 0.17 | 2.2 | 0.35 | 2.6 | 0.39 | 3.4 | 0.76 | 2.2 |
| C18:1 | 1.00 | 12.9 | 4.00 | 30.2 | 3.34 | 29.0 | 15.22 | 43.6 |
| C18:2 | 2.14 | 27.6 | 3.08 | 23.3 | 3.23 | 28.0 | 9.25 | 26.5 |
| C18:3 | 1.80 | 23.6 | 1.88 | 14.2 | 1.51 | 13.1 | 1.72 | 4.9 |
| Total | 7.74 | | 13.23 | | 11.52 | | 34.88 | |

An increase in the total fatty acid content of the nitrogen starved cells was observed, just one day after switching from N-replete medium to N-free medium. This increase was even more pronounced for the cells growing under those conditions at pH 10.0 (up to 35% of dry weight), which may suggest that an additional stress such as high pH could promote lipid accumulation further.

The most abundant fatty acids under nitrogen depletion are the C16:0 and the C18:1. The results also show a relative increase of about two-fold of C18:1 fatty acids under nitrogen deplete conditions for both pH values. Values for microalgal fatty acids content and their accumulation at high pH have never been reported before.

The visualization of the neutral lipid bodies inside the cells was possible using fluorescence microscopy. After a fast diffusion of BODIPY 505/515, the intracellular lipid bodies emitted a strong green fluorescence and could be easily distinguished from the chloroplasts, which appeared in red under the same excitation wavelength. During the course of both experiments the cells showed green fluorescent spherical bodies, either with or without nitrogen in the medium. However, that accumulation was more evident upon nitrogen starvation, with the majority of the cells showing a sharp and strong green fluorescence. FIG. 6 shows the accumulation of those bodies in cells growing under pH 10.0.

The growth of the freshwater N. oleoabundans was investigated under alkaline-saline conditions. A stable cultivation medium was developed under these conditions based on minimal growth requirements and maximal solubility of nutritious salts. An artificial seawater medium with reduced Ca²⁺ and PO₄³⁻ was successful in decreasing mineral precipitation related with high pH levels. The obtained growth rate in this modified medium with 420 mM of salts at pH 10.0 brings a new perspective on the cultivation of microalgae, and in particular N. oleoabundans, and the possible improvement of CO₂ transfer in photobioreactors. Its potential as an alternative source for biofuels production is also shown, as lipids also accumulate under those conditions. Lipid accumulation even seemed more pronounced when nitrogen starvation was applied at high pH.

### Example 4

### Biomass and Fatty Acid Productivity of Neochloris oleabundans Under Alkaline-Saline Conditions

### Experimental Settings

The methods for producing the microalgal strain and growth medium, as well as the photobioreactor are such as described in Example 1.

By varying the dilution rate between 0.6 and 1.9 d−1, two sets of chemostat experiments were carried out: one at reference pH 8.2 and 3.0% salinity, and another at pH 10.0 and 3.4% salinity. The pH was set by the CO₂ supplied via gassing and by the addition of NaHCO₃ and Na₂CO₃ to the medium. At reference pH, 12 mM NaHCO3 was in equilibrium with 1% v/v CO₂. At higher pH, a mixture of 75 mM NaHCO₃ and 75 mM Na₂CO₃ was applied, while gassing with 0.1% v/v CO₂. In this case, the concentration of NaCl in the medium was reduced to 270 mM, as to keep the same osmolarity as in the reference medium.

The cultures were monitored until steady-state was reached. All steady-states were then maintained for 3 to 4 days during which the overflow was collected on ice and harvested every 24 hours for later analysis of biomass dry weight and fatty acids. The microalgae were deprived of nitrogen following some of the nutrient replete steady states, namely after three of the steady-states at reference conditions (dilution rates 0.6 d⁻¹, 1.0 d⁻¹ and 1.9 d⁻¹) and after one steady-state at elevated pH (1.9 d⁻¹). Nitrogen depletion was applied by replacing the feed medium by N-free medium, resulting in a gradual but complete depletion of residual nitrogen in the culture broth. Overflow samples were continued to be collected as described above, for 2 days after medium replacement.

### Biomass Productivity and Observed Yield on Light Energy

The growth medium contained sufficient nutrients to sustain the biomass concentrations obtained in the chemostats. Hence, light availability was the limiting factor for growth (at given temperature, salinity and pH), determining the biomass concentration at the different dilution rates in steady-state. The volumetric biomass productivity (P_{x,vol}, g_{DW} L⁻¹ d⁻¹) was determined daily for each of the steady-states by multiplying the set dilution rate and the measured dry weight biomass concentration in the overflow (Cx, g_{DW} L⁻¹). Under nitrogen depletion, since there was equilibrium with 1% v/v CO₂. At higher pH, a mixture of 75 mM 166 NaHCO3 and 75 mM Na₂CO₃ was applied, while gassing with 0.1% v/v CO₂. In this case, the concentration of NaCl in the medium was no longer a steady-state situation, P_{x,vol} and Cx were determined for one 189 time point only, just before harvest. During photoautotrophic growth, the efficiency of light use can be assessed by the observed biomass yield on light energy (Y_{x/E,obs}, g_{DW} molₚₕ₋₁). This yield can be expressed by Equation (1) as the amount of biomass (g_{DW}) produced per amount of photons absorbed (molₚₕ):

$${Y}_{x / E , obs} = \frac{{P}_{x , vol}}{P {F}_{abs , vol}} = \frac{{C}_{x} \times μ}{{PFD}_{abs} \times ( {A}_{R} / {V}_{R} ) \times 0.001 \times 24 \times 3600 \times {10}^{- 6}} \qquad ( 1 )$$

PF_{abs,vol} (molₚₕ L₋₁ d₋₁) is the photon flux absorbed in a unit volume of the system, i.e. the volumetric photon absorption rate. PF_{abs,vol} corresponds to the product between the photon flux density absorbed by the biomass (PFDabs, according to Equation (2)) and the illuminated surface area (A_{R}, m₂) to volume (V_{R}, m₃) ratio. In Equation (2), PFD_{out,biomass} corresponds to the light passing through the PBR when biomass is present, measured at the PBR's backside at a fixed reference position. Accordingly, PFD_{out,medium} is the light measured at the same reference position with solely medium present.

$${PFD}_{abs} = {PFD}_{in} \times ( 1 - \frac{{PFD}_{out , biomass}}{{PFD}_{out , medium}} ) \qquad ( 2 )$$

### Specific Light Absorption Rate

The specific light absorption rate (r_{E/x}, molₚₕ g_{DW-1} d₋₁) was also determined for each dilution rate in steady-state, as the quotient between the volumetric photon absorption rate in the photobioreactor and the biomass concentration, as described by Equation (3):

$${r}_{E / x = \frac{{PF}_{abs , vol}}{{C}_{x}}} \qquad ( 3 )$$

### Fatty Acids Quantification

Fatty acid methyl esters (FAMEs) were identified and quantified.

Fatty acid productivity (P_{FA}, g_{FA} L₋₁ d₋₁) was determined at each dilution rate in steady state, according to Equation 4:

P_{FA}Pₓ×f_{FA/x} (4)

f_{FA/x} corresponds to the weight fraction of fatty acids in the biomass.

Under nitrogen depletion, since there was no longer a steady-state situation, P_{FA} was determined for one time point only, just before harvest.

### Visualization of Intracellular Neutral Lipid Bodies

The accumulation of intracellular lipid bodies by the microalgae was checked using Confocal Laser Scanning Microscopy (CLSM). The lipophilic fluorescent dye BODIPY 505/515 (4,4-difluoro-1,3,5,7-tetramethyl-4-bora-3a,4a-diaza-sindacene; Invitrogen Molecular Probes, Carlsbad, Calif.) was used for staining the cells.

Slides were prepared for observation under a confocal microscope (Carl Zeiss B.V., Sliedrecht, The Netherlands).

### Effect of the Cultivation Conditions on Biomass Concentration and Productivity

The optimum conditions for the production of N. oleoabundans were evaluated in a photobioreactor operated as a chemostat. We performed two sets of experiments (one at pH 8.2 and another at pH 10.0) in which different dilution rates were applied to assess the maximal productivity at a given incident light intensity. When operating a chemostat under light limiting conditions, the biomass concentration obtained in steady-state is set by the dilution rate applied. This is because the dilution rate equals the specific growth rate of the cells in steady-state. The latter requires a certain amount of light to be available per cell (higher growth rates require more light energy) and thus dictates the attainable biomass concentration at a given incident light intensity.

A decrease of the steady-state biomass concentration (Cx) with increasing dilution rates was observed, varying from 0.89 to 0.39 g_{DW} L₋₁ at pH 8.2 and from 0.60 to 0.22 g_{DW} L₋₁ at pH 10.0 (FIG. 7A). In both cases, the highest Cx was obtained at the lowest dilution rate applied (0.6 d₋₁). The light passing through the system (PFD_{out,biomass}) and the average light intensity in the photobioreactor (PFD_{avg}) in general increased with increasing D (Table 7), as expected due to the decrease in biomass concentration.

TABLE 7
Influence of dilution rate (D, d₋₁) on light
parameters and OD₆₈₀ to OD₇₃₅ ratio. At a given incident photon
flux density ( PFDᵢₙ) , PFD_{out,biomass} is the light passing through
the photobioreactor, PFD_{avg} is the average light intensity
available per cell and PF_{abs} is the light absorbed in the system.

| | D | PFDᵢₙ | PFD_{out,biomass} | PFD_{avg} | PFD_{abs} | |
|---|---|---|---|---|---|---|
| # | (d⁻¹) | μmolₚₕm^{-2s-1} | μmolₚₕm^{-2s-1} | μmolₚₕm^{-2s-1} | μmolₚₕm^{-2s-1} | OD₆₈₀/OD₇₃₅ |
| pH 8.2 | 0.6 ± 0.02 | 427 | 48 ± 0.88 | 173 ± 0.88 | 55 | 2.01 ± 0.01 |
| | 0.7 ± 0.01 | 435 | 70 ± 2.10 | 199 ± 3.87 | 52 | 1.68 ± 0.03 |
| | 1.0 ± 0.01 | 435 | 78 ± 0.19 | 208 ± 0.19 | 51 | 1.92 ± 0.02 |
| | 1.4 ± 0.03 | 433 | 33 ± 1.91 | 153 ± 2.72 | 58 | 2.46 ± 0.03 |
| | 1.4 ± 0.01 | 425 | 38 ± 2.74 | 160 ± 3.64 | 56 | 2.52 ± 0.07 |
| | 1.9 ± 0.004 | 425 | 120 ± 1.79 | 241 ± 1.43 | 44 | 2.08 ± 0.03 |
| pH 10.0 | 0.6 ± 0.01 | 442 | 114 ± 6.51 | 242 ± 5.43 | 46 | 1.85 ± 0.05 |
| | 0.7 ± 0.004 | 442 | 132 ± 5.10 | 257 ± 3.98 | 43 | 1.8 ± 0.01 |
| | 1.0 ± 0.01 | 442 | 140 ± 6.52 | 263 ± 4.96 | 42 | 1.91 ± 0.06 |
| | 1.4 ± 0.01 | 442 | 166 ± 8.04 | 282 ± 5.72 | 38 | 1.98 ± 0.07 |
| | 1.9 ± 0.03 | 442 | 231 ± 2.32 | 325 ± 1.46 | 27 | 1.90 ± 0.03 |

Interestingly, an exception to this trend was observed for the reference pH at D=1.4 d₋₁. Under these conditions, light absorption was the highest of all experiments (experiment performed in separate duplicate runs with similar results, see Table 1), even though the steady-state biomass concentration was markedly lower than those observed for lower dilution rates (FIG. 7A, within FIG. 7 steady state biomass concentration Cx as function of different diluation rates). This higher light absorption was the result of a higher pigmentation of the cells. This is reflected in the significantly higher ratio between OD₆₈₀ and OD₇₃₅ (Table 7), which is an indicator of the chlorophyll content of the biomass. It is not clear though what might be the underlying mechanism governing this trend-breaking adaptation state. We did not observe the same phenomenon at pH 10.0 (FIG. 7A and Table 7), and light absorption data of similar experiments in literature do not show such a phenomenon.

At all dilution rates, the biomass concentration at pH 10.0 was lower than at pH 8.2 (FIG. 7A). This suggests that at pH 10.0 the microalgae need to be exposed to a higher light fraction to realise the same specific growth rate as the cells growing at pH 8.2. The lower biomass concentrations obtained at pH 10.0 thus resulted in lower biomass productivities for each dilution rate (FIG. 7B). For both sets of conditions, an optimal combination of specific growth rate and biomass concentration was observed, resulting in maximal volumetric biomass productivity (FIG. 7B).

The fraction of energy lost to maintenance is lowered when the biomass concentration is reduced, but this increases the culture fraction in which the light intensity is oversaturating for photosynthesis (on the condition that the incident light intensity is oversaturating). Under oversaturating conditions, photosynthesis occurs at maximum rate and the microalgae dissipate the excess of absorbed light energy as heat. At very low biomass concentrations, a large share of the supplied light energy is therefore lost to heat production. In this case, also only a small share is left for biomass formation (sub-optimal productivities at high dilution rates in FIG. 7B). Hence, there is an optimum biomass concentration for which biomass is produced most efficiently with regard to the supplied light energy.

Maximum P_{x,vol} values of 0.98 g_{DW} L₋₁ d₋₁ at reference conditions and 0.47 g_{DW} L₋₁ d₋₁ at higher pH were obtained, both at a dilution rate of 1.4 d₋₁ (FIG. 7B). For the present experimental set-up, this corresponds to optimal biomass concentrations of 0.69 g_{DW} L₋₁ and 0.33 g_{DW} L₋₁, respectively (FIG. 7A).

### Biomass Yield on Light Energy

While the biomass yield on incident light is a very useful parameter for comparison of photobioreactors performance (e.g. different designs, orientation and location), a detailed understanding of the biological conversion of light energy into biomass requires the yield to be expressed per absorbed light energy (Y_{x/E,obs}, Equation 1).

For the reference experiments, Y_{x/E,obs} increased with the dilution rate until 1.4 d₋₁, reaching a value of 0.73 g_{DW} molₚₕ₋₁, and subsequently decreased at the highest dilution rate applied to 0.70 g_{DW} molₚₕ₋₁ (FIG. 8).

At dilution rates below the optimum an increasing portion of the supplied 308 light energy must be used to provide additional ATP for maintenance purposes, either by respiration or possibly by cyclic photophosphorylation. As a consequence, less light energy is available for growth and the biomass yield on light energy drops. The dilution rate at which the volumetric productivity was maximal at pH 8.2 exactly corresponds to the dilution rate where the biomass yield on light energy was the highest. This is as expected since the volumetric light absorption is roughly constant and only the productivity is changing. In contrast, Y_{x/E,obs} did not show a distinct optimum at pH 10.0 and continuously increased with the dilution rate (FIG. 8). In this case, the yield on absorbed light reached a value of 0.63 g_{DW} molₚₕ₋₁ at the highest dilution rate applied (1.9 d₋₁), very close to the one obtained at pH 8.2 at that same dilution rate. All biomass yields at higher pH were lower than those obtained for the same dilution rates at reference pH.

In theory, the lower yields at pH 10.0 could be explained by elevated heat dissipation due to a higher level of oversaturation, by higher energy requirements for biomass formation or for cell maintenance, or a combination of the three. We suggest a higher energy requirement either for biomass formation or for cell maintenance to be the most probable high pH related effects.

The basis for our hypothesis is the relation between the specific light absorption rate (r_{E/x}, molₚₕ d₋₁, Equation 3) and the specific growth rate (μ, d₋₁).

This relation can be described by the model of Pirt (1995) and translated into Equation (5).

$${r}_{E / x} = \frac{μ}{{Y}_{x / E , true}} + {m}_{E / x} \qquad ( 5 )$$

In Equation (5), Y_{x/E,true} is the biomass yield on light energy (i.e. energy required for formation of new biomass, g_{DW} molₚₕ₋₁) and m_{E/x} is the maintenance requirement (molₚₕ g_{DW-1} d₋₁). The model assumes that the fraction of light used by the microalgae for biomass formation increases with p at a constant yield, whilst a fixed amount of light energy per cell is required for cellular maintenance. Plotting the linear regression of μ against r_{E/x} for all steady-states therefore should result in a straight line of which the slope corresponds to the inverse of x/E, true and the offset to m_{E/x}. A non-linear curve at pH 8.2 (FIG. 9A). The lines obtained for both experiments were in this case quite similar, both resulting in a Y x/E, true of 1.09 g_{DW}molₚₕ₋₁. The maintenance requirement was however higher at pH 10.0 (1.3 molₚₕ g_{DW-1}d₋₁ in comparison with 0.8 molₚₕ g_{bio-1} d₋₁ at pH 8.2), which could explain the lower values of Y_{x/E,obs} (FIG. 8). But the non-linear behavior at pH 8.2 is very likely caused 345 by a relatively large loss of energy due to non-photochemical quenching (NPQ) at the highest dilution rate applied. In this case, a higher fraction of light is quenched by heat or fluorescence instead of being absorbed. Such an explanation seems reasonable since the overall biomass yield on absorbed light energy decreased substantially at this dilution rate, while the average light intensity inside the photobioreactor was highest of all dilution rates applied at pH 8.2 (PFD_{avg}, Table 7). This average light intensity was exceeded at all experiments at pH 10.0 (Table 1), especially at the end (D=1.9 d₋₁), so the higher r_{E/x} values could also be due to an increase in NPQ. Moreover, higher r_{E/x} reinforces the idea that individual cells require a higher light availability to grow at the same rates as at pH 8.2. But paradoxically, a non-linear relation between r_{E/x} and D was not observed. In any case, if we assume that in both experimental sets substantial energy losses occurred at D=1.9 d₋₁ due to oversaturation and eliminate the correspondent data points, both regressions will then be linear (FIG. 9B). This alternative scenario gives a mix of 1.1 molₚₕ g_{DW-1} d₋₁ at pH 8.2 and of 1.2 molₚₕ g_{DW-1} d₋₁ at pH 10.0, suggesting that the cultivation pH has hardly an effect on the cellular maintenance requirement. The true biomass yield on absorbed light (Y_{x/E,true}) differs Y substantially though, being then 1.79 g_{DW} molₚₕ₋₁ at pH 8.2 and 0.92 g_{DW} molₚₕ₋₁ at pH 10.0. Hence, we give two possible hypotheses for the reduced overall biomass yield at elevated pH (FIG. 8): increased energy costs for maintenance (FIG. 9A) or for biomass formation (FIG. 9B).

TABLE 8
Comparison of experimental biomass yields on light energy found
in this study with several from other microalgae, at different light
intensities, optimal biomass densities and cultivation pH.

| | | PFD [symbol] | | | |
|---|---|---|---|---|---|
| | Y [symbol] | (μmol [symbol] | C [symbol] | | |
| Organism | (g [symbol] mol [symbol] ⁻¹) | m⁻² s⁻¹) | (g [symbol] L⁻¹) | pH | Reference |
| Neochloris | 0.73^{a}/0.66^{b} | 425 | 0.69 | 8.2 | This study |
| oleoabundans | | | | | |
| Neochloris | 0.63^{a}/0.31^{b} | 442 | 0.33 | 10.0 | This study |
| oleoabundans | | | | | |
| Chlorella | 1.00^{b} | 2100 | 2.10 | 6.7 | Cuaresma |
| sorokiniana | | | | | et al. |
| | | | | | (2009) |
| Dunaliella | 0.65^{b} | 990 | 1.40-3.80 | 7.8 | Zijffers |
| tertiolecta | | | | | et al. |
| | | | | | (2010) |
| Chlamydomonas | 0.80^{b} | 1500 | 1.20^{d} | 7.0 | Kliphuis |
| reinhardtii | | | | | et al. |
| | | | | | (2010a) |
| Neochloris | 0.71^{b} | 270 | 0.90 | 7.5 | Pruvost |
| oleoabundans | | | | | et al. |
| | | | | | (2009) |
| Neochloris | 1.04^{b} | 200 | 0.40 | 7.8 | Sousa |
| oleoabundans | | | | | et al. |
| | | | | | (2012) |
| Chlamydomonas | 0.51^{b,c} | 1000 | 1.10 | 7.5 | Takache |
| reinhardtii | | | | | et al. |
| | | | | | (2010) |
| Chlamydomonas | 0.72^{b,c} | 500 | 0.60 | 7.5 | Takache |
| reinhardtii | | | | | et al |
| | | | | | (2010) |
| Chlamydomonas | 1.11^{b,c} | 110 | 0.35 | 7.5 | Takache |
| reinhardtii | | | | | et al. |
| | | | | | (2010) |

^{a}Y [symbol] based on PFD [symbol] .
^{b}Y [symbol] based on PFD [symbol] .
^{c}Calculated based on Flat-torus PBR experimental data.
^{d}C [symbol] obtained during batch cultivation at the maximum PFD [symbol] applied. [symbol]
indicates data missing or illegible when filed

### Effect of Nitrogen Supply on Biomass Production and Fatty Acid Accumulation

N. oleoabundans was analyzed for its total fatty acid content and composition during all steady-states in nitrogen-replete medium and two days after the medium was replaced with nitrogen-free medium. The biomass productivities, fatty acid content and productivities under nitrogen-replete and depletion conditions are summarized in Table 8.

Despite the pH applied, the total fatty acid content didn't vary substantially within the different dilution rates under nitrogen-replete conditions, being around 8% at pH 8.2 and increasing to 15% at pH 10.0. This higher fatty acid accumulation during growth at higher pH may also explain the lower biomass yields under that condition (sub-section 3.2). It suggests that besides the need for extra light energy for biomass formation or for cell maintenance under less optimal conditions, the cells spend part of that energy for storing more fatty acids.

The constant fatty acid content observed under nitrogen-replete conditions suggests a constant biomass composition. Therefore, the fatty acid productivity followed the same trend as the biomass productivity, by increasing with the dilution rate until reaching a maximum. A maximal volumetric fatty acid productivity of 75.20 mg_{FA} g₋₁ at D=1.4 d₋₁ was found at pH 8.2 and of 66.55 mg_{FA} g₋₁ L₋₁ at pH 10.0 at the same dilution rate.

The higher fatty acid contents obtained at pH 10.0 in general corresponded to higher productivities. Even so, the highest fatty acid productivity was found at pH 8.2, due to the much higher biomass productivity at the corresponding optimal dilution rate.

It has been shown before that nitrogen limitation causes N. oleoabundans cells to stop dividing but, simultaneously, triggers lipid/fatty acid accumulation.

In this case, applying nitrogen depletion therefore caused a decrease of the biomass productivity and a consequent increase of the fatty acid content and productivity. Fatty acids accumulated up to 18% (w/w) when nitrogen depletion was applied at pH 8.2 and their productivity went up to 67 mg_{FA} L₋₁ d₋₁. On the other hand, the total fatty acid content of N. oleoabundans was 29% (w/w) at pH 10.0, corresponding to a productivity of 112.41 mg_{FA} L₋₁ d₋₁, the highest found.

An elevated pH enhances N-starvation-induced fatty acid accumulation. Additionally, pH-related stress by itself can cause an increase on the fatty acid accumulation, being independent from nitrogen-stress. In terms of Neochloris fatty acid composition no variation between the dilution rates applied and pH treatments was observed. An increase of the C16:0 and C18:1 fatty acids upon nitrogen depletion was was observed, especially when associated with elevated pH. In comparison to that, the content of more unsaturated fatty acids was less substantial (FIG. 10), which is more favorable for the preparation of biofuels. The more saturated character of the fatty acids involved requires less hydrogenation during the process. Especially the neutral lipids are of interest, since they are easier to extract and separate.

During the course of both sets of experiments the cells were stained with BODIPY 505/515 and the visualization of neutral lipid bodies was possible, supporting our results. The cells showed green fluorescent spherical bodies, either with or without nitrogen in the medium. The accumulation was more evident after the switch to medium without nitrogen (FIG. 11).

TABLE 9
Total fatty acid content (% FA w/w) and productivity
(P_{FA}, g_{FA} L₋₁ d₋₁) of N. oleoabundans at different dilution
rates (D, d₋₁) at reference conditions (pH 8.2) and higher pH
(pH 10.0). Results without nitrogen limitation are
represented by N(+) and with nitrogen depletion by N(−).
Values of biomass concentration (Cₓ, g_{DW} L₋₁) and productivity
(P_{x,vol}, g_{DW} L₋₁ d₋₁) under the different conditions are also given.

| | | | | | | | PFA, | |
|---|---|---|---|---|---|---|---|---|
| | D | Cₓ | P_{x,vol} (G_{DW} L⁻¹ d⁻¹) | | Total FA (% w/w) | | (mgFA L⁻¹ d⁻¹) | |
| | (d⁻¹) | (GDW L⁻¹) | N(+) | N(−) | N(+) | N(−) | N(+) | N(−) |
| pH 8.2 | 0.6 ± 0.02 | 0.89 | 0.54 ± 0.02 | 0.33 ± 0.01 | 8.74 ± 0.17 | 18.18 ± 0.01 | 46.85 ± 0.93 | 60.52 ± 0.05 |
| | 0.7 ± 0.01 | 0.82 | 0.60 ± 0.01 | — | 8.33 ± 0.002 | — | 49.74 ± 0.01 | — |
| | 1.0 ± 0.01 | 0.78 | 0.77 ± 0.02 | 0.32 ± 0.01 | 7.53 ± 0.01 | 18.52 ± 0.01 | 58.30 ± 0.11 | 64.56 ± 0.03 |
| | 1.4 ± 0.03 | 0.69 | 0.98 ± 0.03 | — | 7.65 | — | 75.20 | — |
| | 1.9 ± 0.004 | 0.39 | 0.73 ± 0.03 | 0.37 ± 0.02 | 7.13 ± 0.01 | 18.11 ± 0.01 | 52.07 ± 0.09 | 67.54 ± 0.02 |
| pH 10.0 | 0.6 ± 0.01 | 0.60 | 0.37 ± 0.01 | — | 14.98 ± 0.01 | — | 54.85 ± 0.01 | — |
| | 0.7 ± 0.004 | 0.52 | 0.38 ± 0.004 | — | 14.89 ± 0.18 | — | 55.84 ± 0.67 | — |
| | 1.0 ± 0.01 | 0.46 | 0.45 ± 0.01 | — | 13.92 ± 0.40 | — | 62.07 ± 1.78 | — |
| | 1.4 ± 0.01 | 0.33 | 0.47 ± 0.01 | — | 14.19 ± 0.03 | — | 66.55 ± 0.12 | — |
| | 1.9 ± 0.03 | 0.22 | 0.41 ± 0.02 | 0.33 ± 0.07 | 14.94 ± 0.01 | 29.20 ± 0.04 | 61.42 ± 0.02 | 112.41 ± 0.17 |

Considering the above, growing N. oleoabundans in alkaline-saline conditions such as in the method according to the present invention, plus nitrogen depletion, brings an advantage in boosting lipid production for microalgal biofuels. Such process can also improve CO₂ transfer in photobioreactors, as less energy will be needed. Further, growing microalgae under alkaline-saline for sustainable production of lipids, carbohydrates and other compounds according to the method of the present invention therefore contributes to cost effective use of microalgae as feedstock for food, fuel, chemicals, and feed at industrial scale. An additional advantage of microalgal growth and lipid production under alkaline-saline conditions such as recited in the present invention is that in these conditions act as an additional hurdle for microbial contamination during the process.

The present invention is by no means limited to the above described preferred embodiments thereof. The rights sought are defined by the following claims within the scope of which many modifications can be envisaged.

1. Method for obtaining algal biomass, comprising the step of culturing algae in a culturing medium having a pH above 8 such that lipid accumulation and/or carbohydrate accumulation in the algal biomass is increased,
wherein the medium is a saline medium, the cultivating medium comprising alkali metal chlorine salts in a concentration of at least 120 mM, wherein carbon dioxide is supplied for carbon dioxide transfer to the medium in a gas which comprises 0.01%-20% (v/v) carbon dioxide.

2. Method according to claim 1, wherein the pH of the medium is in the range of 8-11, preferably 8.5-11, more preferably 9-11 and most preferably 10-11.

3. Method according claim 1, wherein the algal biomass comprises algal cells, preferably lipids comprised by the algal cells.

4. Method according to claim 3, wherein the algae are lipid-rich algae.

5. Method according to claim 1, wherein the algae comprise micro-algae, preferably freshwater micro-algae.

6. Method according to claim 5, wherein the algae are of the species Neochloris oleoabundans.

7. Method according to claim 1, wherein the medium comprises seawater and/or artificial seawater.

8. Method according to claim 1, the cultivating medium comprising alkali metal chlorine salts, preferably NaCl, in a concentration providing the medium with a higher salinity than freshwater, preferably in a concentration of at least 200, 270 or 400 mM.

9. Method according to claim 1, wherein the pH of the culturing medium is regulated by an equilibrium of carbon dioxide, bicarbonate and carbonate.

10. Method according to claim 9, wherein bicarbonate and carbonate ions are present in an amount sufficient to buffer the medium at a pH of at least 8, 8.2, 8.5, 9, 9.5, 10, 10.5 or 11.

11. Method according to claim 1, wherein carbon dioxide is supplied for carbon dioxide transfer to the medium in a gas which comprises 0.038%-20% (v/v) carbon dioxide.

12. Method according to claim 1, wherein the algae are cultivated under conditions of nitrogen limitation, preferably nitrogen starvation.

13. (canceled)

14. Method according to claim 1, wherein the cultivating medium comprises ions of a soluble salt, the concentration being such that no significant precipitation of the ions at the given pH level occurs.

15. Method according to claim 14, wherein the cultivating medium comprises Ca and Mg ions, wherein the concentration of Ca ions is between 0.001 and 0.15 mM and the ratio Ca:Mg is between 1:5 and 1:30, preferably 1:10 to 1:20, more preferably 1:15.

16. (canceled)

17. System for obtaining algal biomass using the method of claim 1, comprising:
a container for holding the cultivating medium; and
means for regulating the pH level of the medium in the container to a level above 8.

18. (canceled)

19. (canceled)
